(19) United States
(12) Patent Application Publication
(10) Pub. No.: US 2011/0190901 A1
(43) Pub. Date: Aug. 4, 2011

(54) Acetabular Prosthetic Devices and Associated Methods

(75) Inventors: Noam Weissberg, Rishon-Lezion (IL); Avraham Shterling, Yarkona (IL); Gal Zur, Yarkona (IL)

(73) Assignee: ACTIVE IMPLANTS CORPORATION, Memphis, TN (US)

(21) Appl. No.: 12/699,093

(22) Filed: Feb. 3, 2010

(51) Int. Cl. A61F 2/34

(52) U.S. Cl. 623/22.24

(57) ABSTRACT

Devices, apparatus, and systems for replacing at least some of the functionality of the natural hip joint and associated methods of implantation are disclosed. In one aspect a prosthetic acetabular cup system is provided. The system includes a metal shell comprising an outer surface for securely engaging a prepared portion of an acetabulum and an opposing inner surface. In some instances, portions of the inner and outer surfaces define an anchoring section that is deformable between an insertion configuration—where the anchoring section projects inwardly from the inner surface—and an anchoring configuration—where the anchoring section projects outwardly from the outer surface to define an anchoring protrusion for engaging the prepared portion of the acetabulum. In some embodiments, the system also includes a pliable articulating component having an outer surface including at least one engagement feature sized and shaped to engage with the metal shell. The pliable articulating component includes an inner surface for articulatingly receiving a femoral head.

### TECHNICAL FIELD

Embodiments of the present disclosure relate generally to medical prosthetic devices, including prosthetic hip joint components, and associated methods of implantation and treatment.

### BACKGROUND

The present disclosure relates to devices, apparatus, and systems for replacing at least some of the functionality of the natural hip joint and methods of implanting such devices, apparatus, and systems. The natural hip joint is a ball-and-socket joint formed by the articulating interaction of the rounded head of the femur with the acetabulum of the pelvis. The articulating surfaces of both the head of the femur and the acetabulum are covered with articular cartilage. Various conditions can cause damage to the hip joint resulting in debilitating pain, arthritis, and/or limited mobility. In some instances, hip arthroplasty has been used to treat such conditions.

Although existing devices and methods associated with prosthetic hip joint components have been adequate in some respects, they have not been satisfactory in all respects. The present disclosure overcomes one or more of the shortcomings of the existing devices and methods.

### SUMMARY

In one embodiment, a prosthetic device for positioning within a hip joint is disclosed.

In some instances, the prosthetic device is comprised of a metal shell component and a polymer liner component. The metal shell includes a convex outer surface for securely engaging a prepared portion of an acetabulum and an opposing concave inner surface for receiving a polymer liner component. A majority of the outer surface has a generally semi-spherical profile and a majority of the inner surface has a generally semi-spherical profile concentric with the outer surface. At least a portion of the inner and outer surfaces define an anchoring section extending circumferentially about the metal shell component between the apex and rim of the metal shell component. The anchoring section is deformable between an insertion configuration and an anchoring configuration. In the insertion configuration the anchoring section projects inwardly from the inner surface and, in the anchoring configuration, the anchoring section projects outwardly from the outer surface to define an anchoring protrusion for engaging the prepared portion of the acetabulum. A majority of the metal shell component has a substantially uniform thickness between the outer surface and the inner surface, while the anchoring section has a thickness between the outer surface and the inner surface that is less than the substantially uniform thickness of the majority of the metal shell component. The polymer liner component includes an outer surface having a generally semi-spherical profile for engagement with the inner surface of the metal shell component. The outer surface includes an annular protrusion extending circumferentially about the outer surface that is sized and shaped to engage a recess defined by the inner surface of the anchoring section of the metal shell component when the anchoring section is in the anchoring configuration. The polymer liner component also includes an inner surface for articulatingly mating with a femoral head.

In another embodiment, a method of implanting a prosthetic device for positioning within a hip joint is disclosed.

In some instances, a method of implanting a prosthetic device includes preparing a portion of an acetabulum to receive a hip prosthesis and obtaining a hip prosthesis suitable for insertion into the prepared acetabulum. In one embodiment, the hip prosthesis includes a metal shell component and a polymer liner component. The metal shell component includes a convex outer surface for securely engaging the prepared portion of the acetabulum and an opposing concave inner surface for receiving a polymer liner component. A majority of the outer surface has a generally semi-spherical profile and a majority of the inner surface having a generally semi-spherical profile concentric with the outer surface. At least a portion of the inner and outer surfaces define an anchoring section extending circumferentially about the metal shell component between the apex and rim of the metal shell component. The anchoring section is deformable between an insertion configuration where the anchoring section projects inwardly from the inner surface and an anchoring configuration where the anchoring section projects outwardly from the outer surface. The polymer liner component includes an outer surface having a generally semi-spherical profile for engagement with the inner surface of the metal shell component. The outer surface includes an annular protrusion extending circumferentially about the outer surface. The polymer liner component also includes an inner surface for articulatingly mating with a femoral head. The method also includes inserting the metal shell component of the hip prosthesis into the prepared portion of the acetabulum with the anchoring section in the insertion configuration and deforming the anchoring section of the metal shell component from the insertion configuration to the anchoring configuration to securely fix the metal shell component to the prepared portion of the acetabulum by engagement of the anchoring section with the prepared portion of the acetabulum. Finally, the method includes inserting the polymer liner component such that the outer surface of the polymer liner component engages the inner surface of the metal shell component.

In another embodiment, a method of implanting a prosthetic device includes inserting a metal shell into a prepared acetabulum, where the metal shell includes a convex outer surface and an opposing concave inner surface. A majority of the outer surface has a partially spherical profile and a majority of the inner surface has a partially spherical profile substantially concentric with the majority of the outer surface. At least a portion of the inner and outer surfaces define an anchoring section, the anchoring section deformable between an insertion configuration where the anchoring section projects inwardly relative to the partially spherical profile of the outer surface and an anchoring configuration where the anchoring section projects outwardly relative partially spherical profile of the outer surface. The metal shell is inserted into the prepared acetabulum with the anchoring section in the insertion configuration. The method further comprises deforming the anchoring section of the metal shell component from the insertion configuration to the anchoring configuration to secure the metal shell to the prepared acetabulum by engagement of the anchoring section with the prepared acetabulum.

### BRIEF DESCRIPTION OF DRAWINGS

Other features and advantages of the present disclosure will become apparent in the following detailed description of embodiments of the disclosure with reference to the accompanying of drawings, of which:

FIG. 1 is a perspective view a prosthetic device according one embodiment of the present disclosure.

FIG. 2 is a cross-sectional side view of the prosthetic device of FIG. 1.

FIG. 3 is a perspective view of a shell component of the prosthetic device of FIGS. 1 and 2 in an insertion configuration, according to one embodiment of the present disclosure.

FIG. 4 is a cross-sectional side view of the shell component of FIG. 3 in the insertion configuration.

FIG. 5 is a cross-sectional side view of the shell component of FIGS. 3 and 4 in a transitional configuration.

FIG. 6 is a cross-sectional side view of the shell component of FIGS. 3-5 in an anchoring configuration.

FIG. 7 is a perspective view of the shell component of FIGS. 3-6 in the anchoring configuration.

FIG. 8 is a perspective view of a patient's prepared acetabulum according to one aspect of the present disclosure.

FIG. 9 is a perspective view of the prosthetic device of FIGS. 1 and 2, including the shell component of FIGS. 3-7, and the patient's prepared acetabulum of FIG. 8 illustrating a first stage of implantation of the prosthetic device into the patient's prepared acetabulum according to one aspect of the present disclosure.

FIG. 10 is a perspective view of the prosthetic device and the patient's prepared acetabulum similar to that of FIG. 9, but showing a second stage of implantation of the prosthetic device into the patient's prepared acetabulum.

FIG. 11 is a perspective view of the prosthetic device and the patient's prepared acetabulum similar to that of FIGS. 9 and 10, but showing the prosthetic device fully implanted into the patient's prepared acetabulum.

FIG. 12 is a cross-sectional side view of a portion of the shell component of FIGS. 3-7 and a portion of the patient's prepared acetabulum of FIG. 8 illustrating insertion of the shell component into the patient's prepared acetabulum in the insertion configuration.

FIG. 13 is a cross-sectional side view of the portion of the shell component and the portion of the patient's prepared acetabulum similar to that of FIG. 12, but illustrating the shell component in the transitional configuration.

FIG. 14 is a cross-sectional side view of the portion of the shell component and the portion of the patient's prepared acetabulum similar to that of FIGS. 12 and 13, but illustrating the shell component in the anchoring configuration.

FIG. 15 is a cross-sectional side view of a portion of the prosthetic device and a portion of the patient's prepared acetabulum illustrating the prosthetic device fully implanted within the patient's prepared acetabulum.

FIG. 16 is a cross-sectional side view of the prosthetic device of FIGS. 1 and 2 and the patient's prepared acetabulum of FIG. 8 illustrating an initial phase of insertion of the prosthetic device into the patient's prepared acetabulum according to one aspect of the present disclosure.

FIG. 17 is a cross-sectional side view of the prosthetic device and the patient's prepared acetabulum similar to that of FIG. 16, but illustrating another phase of insertion of the prosthetic device into the patient's prepared acetabulum.

FIG. 18 is a cross-sectional side view of the prosthetic device and the patient's prepared acetabulum similar to that of FIGS. 16 and 17, but illustrating the prosthetic device fully implanted within the patient's prepared acetabulum.

FIG. 19 is a cross-sectional side view of a shell component according to another aspect of the present disclosure positioned within a patient's prepared acetabulum in an insertion configuration.

FIG. 20 is a cross-sectional side view of the shell component of FIG. 19 anchored within the patient's prepared acetabulum.

FIG. 21 is a cross-sectional side view of an insertion tool being utilized to transition a shell component positioned within a patient's prepared acetabulum from an insertion configuration to a transition configuration according to another aspect of the present disclosure.

FIG. 22 is a cross-sectional side view of another insertion tool being utilized to transition a shell component positioned within a patient's prepared acetabulum from the transition configuration to an anchored configuration according to another aspect of the present disclosure.

FIG. 23 is a cross-sectional side view similar to that of FIG. 22, but illustrating the shell component in the anchored configuration.

FIG. 24 is a top view of the insertion tool of FIGS. 22 and 23.

FIG. 25 is a cross-sectional side view of an insertion tool being utilized to transition a shell component positioned within a patient's prepared acetabulum from an insertion configuration to an anchored configuration according to another aspect of the present disclosure.

FIG. 26 is a bottom view of the insertion tool of FIG. 25.

FIG. 27 is a perspective view of the insertion tool of FIGS. 25 and 26.

### DETAILED DESCRIPTION

For the purposes of promoting an understanding of the principles of the present disclosure, reference will now be made to the embodiments illustrated in the drawings, and specific language will be used to describe the same. It will nevertheless be understood that no limitation of the scope of the disclosure is intended. Any alterations and further modifications in the described devices, instruments, methods, and any further application of the principles of the disclosure as described herein are contemplated as would normally occur to one skilled in the art to which the disclosure relates. In particular, it is fully contemplated that the features, components, and/or steps described with respect to one embodiment may be combined with the features, components, and/or steps described with respect to other embodiments of the present disclosure.

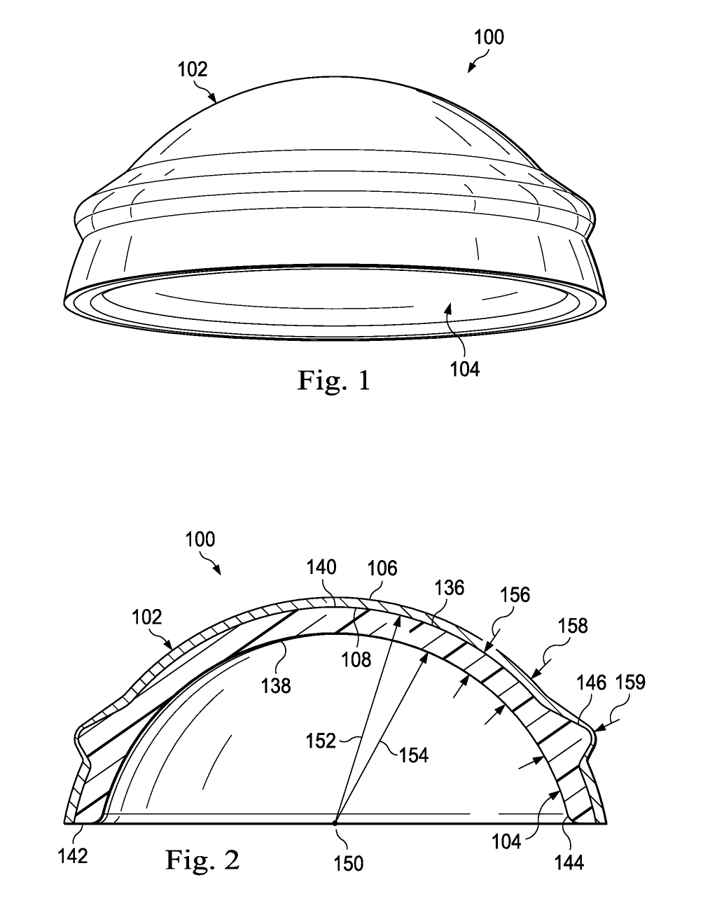

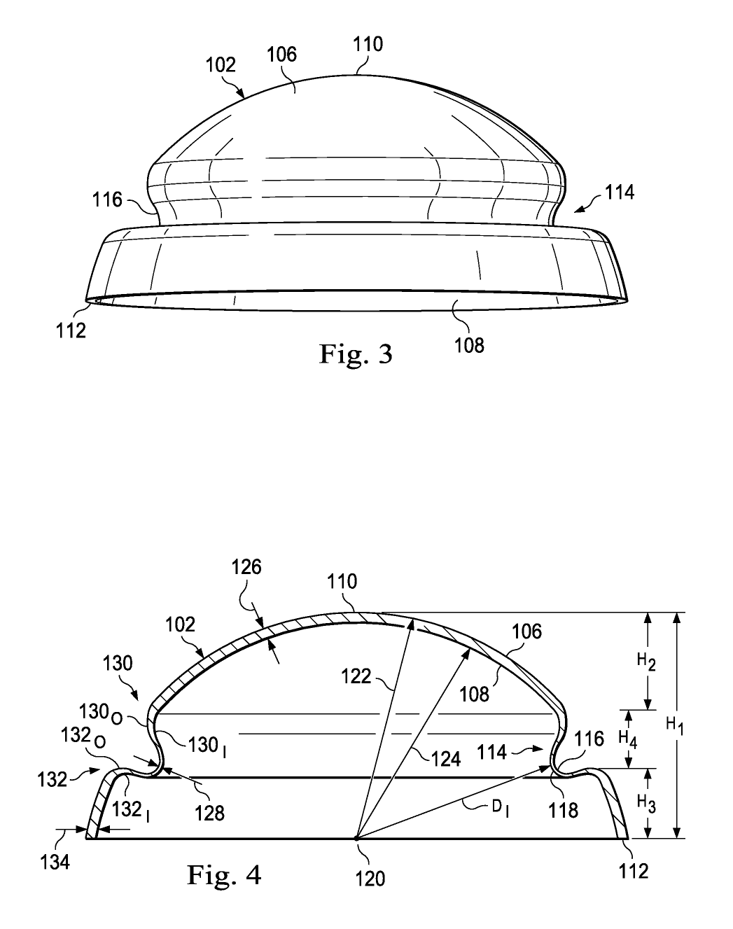

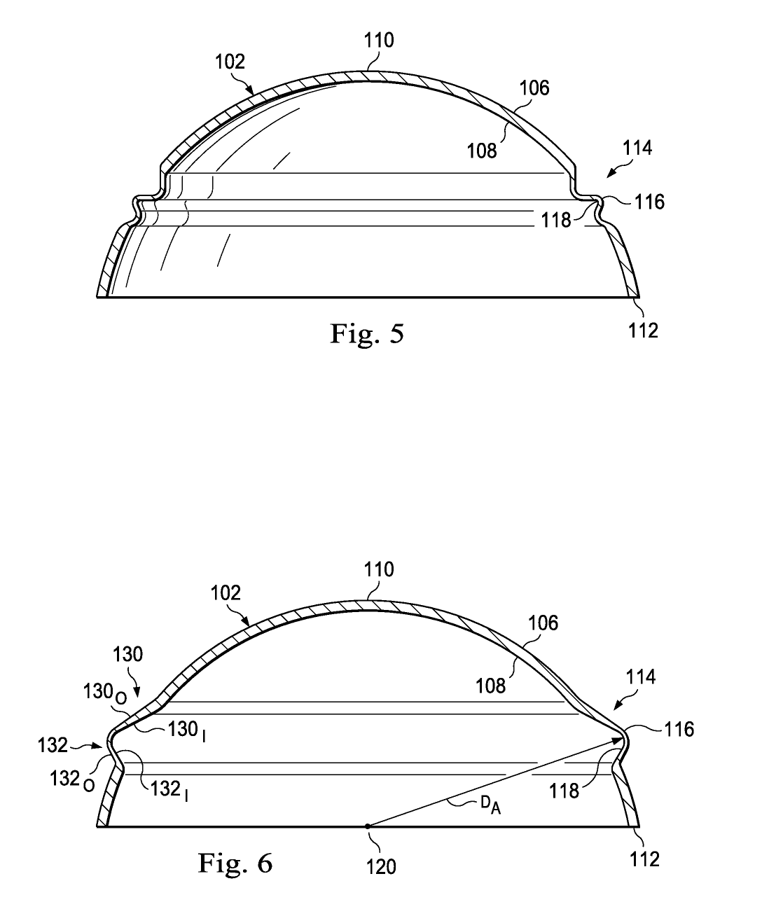

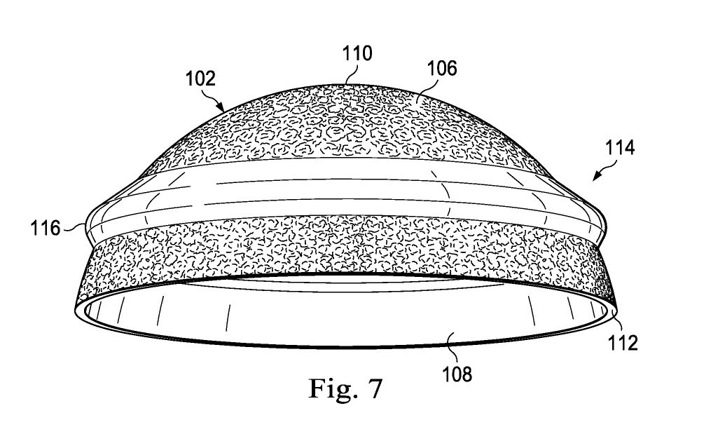

Referring now to FIGS. 1, 2, 3, 4, 5, 6, and 7, shown therein are aspects of a prosthetic device 100 according to one embodiment of the present disclosure. The prosthetic device 100 includes at least a shell component 102 and a liner component 104. FIG. 1 is a perspective view the prosthetic device 100; FIG. 2 is a cross-sectional side view of the prosthetic device 100; FIG. 3 is a perspective view of the shell component 102 in an insertion configuration; FIG. 4 is a cross-sectional side view of the shell component 102 in the insertion configuration; FIG. 5 is a cross-sectional side view of the shell component 102 in a transitional configuration; FIG. 6 is a cross-sectional side view of the shell component 102 in an anchoring configuration; and FIG. 7 is a perspective view of the shell component 102 in the anchoring configuration.

Referring more specifically to FIGS. 3-7, the shell component 102 includes an outer surface 106 for engaging a prepared portion of a patient's acetabulum. In that regard, in some instances, the outer surface 106 of the shell component 102 is treated to enhance engagement between the outer surface and the patient's acetabulum. For example, FIG. 7 illustrates one embodiment where the outer surface 106 has been treated to enhance engagement with the patient's acetabulum. In some instances, the outer surface 106 is roughened to increase the friction between the acetabulum and the shell component 102. Further, the outer surface 106 may be treated with biologics to encourage ingrowth of bone and/or articular cartilage. In some instances, the engagement surface receives one or more surface treatments as described in U.S. patent application Ser. No. 10/497,897 titled “CUSHION BEARING IMPLANTS FOR LOAD BEARING APPLICATIONS,” hereby incorporated by reference in its entirety. Further, in some instances the outer surface 106 includes structural features for encouraging engagement between the shell component 102 and the acetabulum. For example, the outer surface 106 includes projections, recesses, and/or combinations thereof in some instances. In the illustrated embodiment, the shell component 102 includes an anchoring section 114 that is described in greater detail below. Generally, the outer surface 106 may be fixedly secured to the acetabulum in any medically suitable manner. In that regard, the outer surface 106 may engage a bony portion of the acetabulum, articular cartilage of the acetabulum, and/or combinations thereof.

Referring more specifically to FIGS. 3 and 4, the shell component 102 is shown in an insertion configuration. In that regard, the anchoring section 114 is deformed inwardly to facilitate insertion of the shell component 102 into a prepared acetabulum. As shown, the anchoring section 114 is defined by a portion 116 of the outer surface 106 and a portion 118 of the inner surface 108. The anchoring section is deformable or movable between the insertion configuration (illustrated in FIGS. 3 and 4) and an anchoring configuration (illustrated in FIGS. 6 and 7).

As shown in FIG. 4, in the present embodiment a majority of the outer surface 106 has a generally semi-spherical profile. Similarly, a majority of the inner surface 108 also has a generally semi-spherical profile that is concentric with the semi-spherical profile of the outer surface 106. From a center point 120, the majority of the outer surface 106 is defined by a radius of curvature 122, while the majority of the inner surface 108 is defined by a radius of curvature 124. As illustrated, the majority of the outer surface 106 and the majority of the inner surface 108 are separated by a thickness 126. Generally, the thickness 126 is between about 0.1 mm and about 3.0 mm and, in some embodiments, is between about 0.1 mm and about 1.5 mm. In one particular embodiment, the thickness is approximately 1.0 mm. The radius of curvature 122 is generally between about 20 mm and about 34 mm, but in some instances may be larger or smaller. Accordingly, the radius of curvature 124 is generally between about 19.9 mm and about 33.9 mm, but also may be larger or smaller in some instances. In some embodiments, the center point for the radius of curvature 124 of the inner surface 108 is offset with respect to the center point for the radius of curvature 122 of the outer surface 106 such that the inner surface defines less than a full hemi-spherical profile that spans 180°. For example, in some instances, the inner surface 108 defines a partially-spherical profile that spans between about 150° and about 180° of a spherical profile and, in one particular embodiment, spans about 160°.

In the illustrated embodiment, the anchoring section 114 has a thickness 128 between the portions 116, 118 of the outer and inner surfaces 106, 108 defining the anchoring section. The thickness 128 of the anchoring section 114 is less than or equal to the thickness 126. The thickness 128 is typically between about 0.1 mm and about 1.5 mm and, in some embodiments, is between about 0.1 mm and about 1.0 mm. In one particular embodiment, the thickness is approximately 0.3 mm. The shell component 102 includes a transition 130 between the anchoring section 114 and the upper portion of the shell component positioned between the anchoring section and the apex 110. In that regard, the transition 130 includes an outer portion 130_{O} defined by the transition between an upper portion of the outer surface 106 and the portion 116 of the outer surface defining the anchoring section 114 and an opposing inner portion 130_{I} defined by the transition between an upper portion of the inner surface 108 and the portion 118 of the inner surface defining the anchoring section 114. In the illustrated embodiment, the thickness 128 is illustrated as being approximately half of the thickness 126. Accordingly, in the illustrated embodiment the transition 130 is a taper that facilitates the change in thickness. The transition 130 is illustrated as being a gradual taper, but in other embodiments the taper is steeper and, in some instances, is abrupt such that an edge or step is defined by the change in thickness. As discussed below with respect to the functionality of the anchoring section, in some instances the transition 130 to the anchoring section 114 is defined by a change in the material properties of the shell component 102. In that regard, in some embodiments, the thickness 128 of the anchoring section 114 is substantially equal to the thickness 126, but the material properties of the anchoring section 114 are different from those of at least the upper portion of the shell component 102 between the anchoring section and the apex 110. In some embodiments, the transition 130 includes both a change in thickness as well as a change in material properties.

Similarly, the shell component 102 includes a transition 132 between the anchoring section 114 and the lower portion of the shell component positioned between the anchoring section and the rim 112. In that regard, the transition 132 includes an outer portion 132_{O} defined by the transition between a lower portion of the outer surface 106 and the portion 116 of the outer surface defining the anchoring section 114 and an opposing inner portion 132_{I} defined by the transition between a lower portion of the inner surface 108 and the portion 118 of the inner surface defining the anchoring section 114. The lower portion of the shell component 102 positioned between the anchoring section 114 and the rim 112 has a thickness 134 between the outer surface 106 and the inner surface 108. The thickness 134 is generally equal to or greater than the thickness 126 and, therefore, is also generally equal to or greater than the thickness 128. In that regard, the thickness 134 is typically between about 0.1 mm and about 2.0 mm and, in some embodiments, is between about 0.1 mm and about 0.6 mm. In one particular embodiment, the thickness is approximately 0.3 mm.

In some instances, the thickness 134 of the lower portion of the shell component 102 is increased relative to the thickness 128 of the anchoring section 114 to prevent unwanted deformation of the shell component during transition or deformation of the anchoring section from the insertion configuration of FIGS. 3 and 4 to the anchoring configuration of FIGS. 6 and 7. In other instances, the shell component 102 includes a reinforcing ring or band around, within, and/or inside the lower portion of the shell component to limit deformation of the shell component between the anchoring section 114 and the rim 112. In some instances, the material properties of the lower portion of the shell component 102 are different from those of at least the anchoring section 114. Thus, in some embodiments the transition 132 between the anchoring section 114 and the lower portion of the shell component 102 is defined by a change in the material properties of the shell component 102. In that regard, in some embodiments, the thickness 134 is substantially equal to the thickness 128 of the anchoring section 114, but the material properties of the anchoring section 114 are different from those of at least the lower portion of the shell component 102 between the anchoring section and the rim 212. In some embodiments, the transition 132 includes both a change in thickness as well as a change in material properties. In the illustrated embodiment, the thickness 134 is illustrated as being approximately twice the thickness 128. Accordingly, the transition 132 is illustrated as a taper that facilitates the change in thickness. The transition 132 is illustrated as being a gradual taper, but in other embodiments the taper is steeper and, in some instances, is abrupt such that an edge or step is defined by the change in thickness.

The shell component 102 also has a height H₁ between the apex 110 of the shell component and the rim 112. In that regard, in embodiments where the shell component 102 is substantially semispherical the height H₁ is substantially equal to the radius of curvature 122 of the outer surface 106. The shell component 102 also has a height H₂ between the apex 110 of the shell component and the transition 130 to the anchoring section 114. Similarly, the shell component 102 has a height H₃ between the rim 112 of the shell component and the transition 132 to the anchoring section 114. Accordingly, the anchoring section 114 has a height H₄ that is equal to the height H₁ minus the heights H₂ and H₃. In some embodiments, the height H₁ is between about 24.0 mm and about 30.0 mm, the height H₂ is between about 6.0 mm and about 18.0 mm, the height H₃ is between about 2.0 mm and about 8.0 mm, and the height H₄ is between about 3.0 mm and about 10.0 mm.

Referring now generally to FIGS. 4-6, the function of the anchoring section 114 will be discussed in greater detail. As a general matter, the anchoring section 114 is movable between an insertion configuration where the anchoring section projects inward relative to the outer surface 106 of the shell component 102 (as shown in FIG. 4) and an anchoring configuration where the anchoring section projects outward relative to the outer surface of the shell component (as shown in FIG. 6). In the transition between the insertion configuration and the anchoring configuration the anchoring section 114 is deformed through a transition configuration (as shown in FIG. 5).

Referring more specifically to FIG. 4, when in the insertion configuration the anchoring section 114 projects inward relative to the outer surface 106 such that it defines a recess or depression in the outer surface 106 and defines a corresponding projection or protrusion in the inner surface 108. As shown, in the insertion configuration the outer portions 130_{O} and 132_{O} of the transitions 130 and 132 each define a convex surface, while the inner portions 130_{I} and 132_{I} of the transitions 130 and 132 each define a concave surface. Further, in the insertion configuration the portion 118 of the inner surface 108 defining the anchoring section 114 is spaced from the center point 120 by a distance D_{I}. As shown, the distance D_{I} is less than the radius of curvature 124 defining the majority of the inner surface 108 such that the anchoring section 114 is retracted radially relative the majority of the inner surface 108. In that regard, in some instances, the distance D_{I} is between about 15.0 mm and about 29.0 mm. Having the anchoring section 114 retracted radially relative to the inner surface 108 and, therefore, the outer surface 106 (in contrast to the anchoring configuration where the anchoring section protrudes radially beyond the outer surface) facilitates safe insertion of the shell component 102 into a prepared acetabulum. In that regard, if the shell component 102 was inserted with the anchoring section 114 protruding radially outward from the outer surface, the anchoring section could cause damage to the acetabulum as the shell component is urged into the acetabulum. By having the anchoring section 114 projecting inwardly in the insertion configuration, the potential for damaging the prepared acetabulum during insertion of the shell component 102 is greatly reduced or eliminated completely.

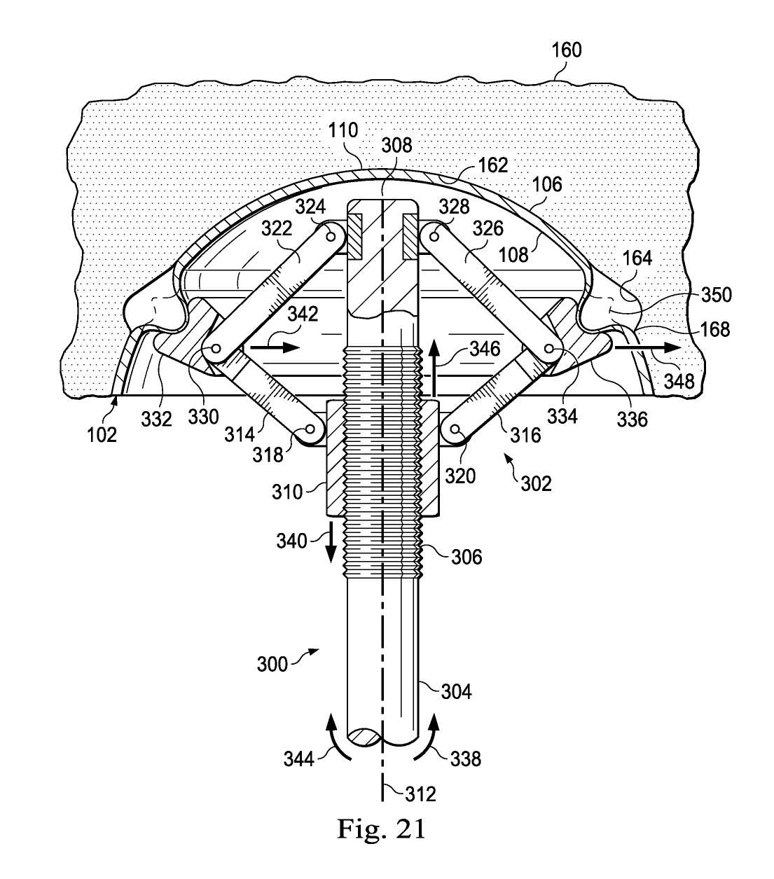

From the insertion configuration, the anchoring section 114 is moved to the anchoring configuration. The transition between the insertion configuration and the anchoring configuration is achieved through mechanical force in some instances. For example, a mechanical tool is utilized to urge the anchoring section 114 from the insertion configuration to the anchoring configuration. Exemplary embodiments of some surgical tools suitable for transitioning the anchoring section from the insertion configuration to the anchoring configuration are described below with respect to FIGS. 21-27. However, it is understood that these tools are merely examples of suitable tools and do not limit the types of tools that are suitable for use with the shell component 102 in any way.

In other instances, the transition between the insertion configuration and the anchoring configuration is achieved by transitioning a shape-memory material between a first material state and a second material state. In that regard, the anchoring section 114 comprises a shape-memory material, such as Nitinol, in some embodiments. The shape-memory material is configured such that in the first state the shape-memory material is biased to the insertion configuration and in the second state the shape-memory material is biased to the anchoring configuration. In some instances, the transition of the shape-memory material between the first and second states and, thereby, the insertion and anchoring configurations is achieved by changing the temperature of the shape-memory material. In some embodiments, the shape-memory material is biased towards the second state (i.e., the anchoring configuration) when the shape-memory material is between about 35° C. and about 40° C.

In the transition between the insertion configuration and the anchoring configuration, the anchoring section 114 will pass through a transition configuration, such as that shown in FIG. 5. The transition configuration shown in FIG. 5 is only one example of a transition configuration and illustrates only a single point in the transition between the insertion configuration and the anchoring configuration. In that regard, it is understood that the actual shape profile of the anchoring section 114 at any particular point during the transition between the insertion configuration and the anchoring configuration is dependent upon several factors, including but not limited to the structural and/or material properties of the anchoring section 114, the structural and/or material properties of the surrounding portions of the shell component 102, the profile of the anchoring section 114 when in the insertion configuration, the profile of the anchoring section 114 when in the anchoring configuration, and/or the manner in which the anchoring section 114 is being moved between the insertion and anchoring configurations.

Referring more specifically to FIG. 6, once the anchoring section 114 has been fully transitioned to the anchoring configuration the anchoring section 114 projects outward relative to the outer surface 106 such that it defines projection or protrusion extending from the outer surface 106 and defines a corresponding recess or depression in the inner surface 108. As shown, in the anchoring configuration the outer portions 130_{O} and 132_{O} of the transitions 130 and 132 each define a concave surface, while the inner portions 130_{I} and 132_{I} of the transitions 130 and 132 each define a convex surface. Further, in the anchoring configuration the portion 118 of the inner surface 108 defining the anchoring section 114 is spaced from the center point 120 by a distance D_{A}. As shown, the distance D_{A} is greater than the radius of curvature 122 defining the majority of the outer surface 106 such that the anchoring section extends radially beyond the majority of the outer surface 106. In that regard, in some instances, the distance D_{A} is between about 25.0 mm and about 34.0 mm Having the anchoring section 114 extending radially beyond the outer surface 106 (in contrast to the insertion configuration) facilitates engagement of the shell component 102 with a prepared acetabulum. In that regard, in some instances the anchoring section 114 is particularly suited to fixedly engage a recess prepared in the acetabulum. The engagement between the protrusion defined by the anchoring section 114 and the recess in the acetabulum fixedly secures the shell component 102 within the acetabulum and prevents unwanted loosening and/or removal of the shell component from the acetabulum.

Referring again to FIG. 2, the liner component 104 includes an outer surface 136 shaped to mate with the inner surface 108 of the shell component 102. The outer surface 136 includes an anchoring protrusion 146 sized and shaped to engage the recess defined by the anchoring section 114 of the shell component 102 when the anchoring section 114 is in the anchoring configuration shown in FIGS. 6 and 7. In that regard, the anchoring protrusion 146 has a generally rounded profile that is substantially the inverse of the profile of the recess defined by the anchoring section 114 when the anchoring section 114 is in the anchoring configuration. Generally, the engagement between the anchoring protrusion 146 and the anchoring section 114 secures the liner component 104 within the shell component 102 and constrains movement of the liner component relative to the shell component. In some instances, the anchoring protrusion 146 snap-fits into the recess defined by the anchoring section 114. In that regard, in some embodiments the liner component 104 is inserted into the shell component 102 after the anchoring section 114 has been transitioned to the anchoring configuration. In other embodiments, the liner component 104 is inserted into the shell component 102 with the anchoring section 114 in the insertion configuration such that as the liner component is advanced into the shell component the anchoring protrusion 146 interfaces with the anchoring section 114 and urges the anchoring section outward to the anchoring configuration. In such embodiments, movement of the anchoring section 114 outward to the anchoring configuration also results in secure engagement of the liner component 104 to the shell component 102 via the engagement of the anchoring protrusion 146 with the recess defined by the anchoring section 114 being in the anchoring configuration.

The liner component 104 also includes an inner surface 138 opposite the outer surface 136. Generally, the inner surface 138 is configured for articulatingly mating with a femoral head. In some instances, the femoral head is a prosthetic component. In other instances, the femoral head is a natural femoral head. In that regard, in some instances a natural femoral head is shaped or conditioned for use with the liner component 104. For example, portions of the natural femoral head may be removed to shape the femoral head such that the resulting femoral head defines an articulating surface that substantially matches the inner surface 138 of the liner component 104.

In the present embodiment, a majority of the outer surface 136 has a generally semi-spherical profile. Similarly, a majority of the inner surface 138 also has a generally semi-spherical profile that is concentric with the semi-spherical profile of the outer surface 136. From a center point 150, the majority of the outer surface 136 is defined by a radius of curvature 152, while the majority of the inner surface 138 is defined by a radius of curvature 154. As illustrated, the majority of the outer surface 136 and the majority of the inner surface 138 are separated by a thickness 156. Generally, the thickness 156 is between about 1.0 mm and about 6.0 mm and, in some embodiments, is between about 2.0 mm and about 4.0 mm. In one particular embodiment, the thickness is approximately 3.0 mm. The radius of curvature 152 is generally between about 19.0 mm and about 33.9 mm, but in some instances may be larger or smaller. Accordingly, the radius of curvature 154 is generally between about 15.0 mm and about 27.0 mm, but also may be larger or smaller in some instances. In some embodiments, the center point for the radius of curvature 154 of the inner surface 138 is offset with respect to the center point for the radius of curvature 152 of the outer surface 136 such that the inner surface defines less than a full semi-spherical profile, which would normally span 180°. For example, in some instances, the inner surface 138 defines a partially-spherical profile that spans between about 150° and about 180° of a spherical profile and, in one particular embodiment, spans about 160°.

In some instances, the liner component 104 includes deformation control elements or reinforced material adjacent to and/or defining the anchoring protrusion 146. In that regard, the deformation control elements and/or the reinforced material can strengthen the structural integrity of the liner component 104 to prevent unwanted interruption to the inner articulating surface 138 that may be caused by heavy loading of the hip joint distributed through the anchoring section 114 of the shell component 102 and into the liner component 104 through the anchoring protrusion 146.

As shown in FIG. 2, the shell component 102 and the liner component 104 are securely engaged with one another. In the present embodiment, the liner component 104 is snap-fit within the shell component 102. In that regard, the anchoring protrusion 146 of the liner component 104 snap-fits into the recess defined by the anchoring section 114 of the shell component 102. In some instances, engagement of the liner component 104 with the shell component 102 causes the liner component 104 to deform as it is positioned or inserted within the shell component 102. Specifically, portions of the liner component 104, including its outer and inner surfaces 136, 138, are deformed inwardly such that the outer and inner surfaces are not partially spherical. Instead, the outer and inner surfaces 136, 138 become partially elliptical or oblong in some instances. For example, in some instances the apex 140 of the outer surface 136 of the liner component 104 is positioned closer to the inner surface 108 of the shell component 102 than remaining portions of the outer surface 136 until the anchoring protrusion 146 is received within the recess defined by the anchoring section 112.

When assembled, the shell component 102 and the liner component 104 have a combined thickness 158 between the outer surface 106 of the shell component 102 and the inner surface 138 of the liner component 104. In the illustrated embodiment, the thickness 158 is substantially constant about a majority of the prosthetic device 100. In that regard, the thickness of prosthetic device 100 varies relative to the thickness 158 due to profiles of the anchoring section 114 of the shell component 102 and the anchoring protrusion 146 of the liner component 104. For example, adjacent to the engagement of the anchoring section 114 and the anchoring protrusion the prosthetic device 100 has a maximum thickness 159, which is greater than the thickness 158 of the majority of the prosthetic device. In some embodiments, the thickness 159 is between about 4.0 mm and about 10.0 mm and, in one particular embodiment, is about 6.0 mm. In other embodiments, the combined thickness extending between the outer surface 106 and the inner surface 138 is not substantially constant about a majority of the prosthetic device. In one particular embodiment, the thickness of the prosthetic device 100 adjacent to the rim of the components 102, 104 is larger or thicker than the thickness adjacent the apex of the components. In that regard, an increased thickness adjacent to the rim of the components is utilized to increase the structural strength of the prosthetic device adjacent to the rim and limit deformation of the rims of the components. In some instances, the increased thickness adjacent to the rim is utilized to retain the femoral head within the articulating component in some instances.

While the shell component 102 is shown as having anchoring section 114, in other embodiments the shell component may have other engagement features for mating the with the acetabulum and/or the liner component 104. Similarly, while the liner component 104 is shown as having anchoring protrusion 146, in other embodiments the liner component may have other engagement features for mating with the shell component 102. In that regard, each of the shell component 102 and the articulating component 104 may include projections, recesses, and combinations thereof sized and shaped to engage corresponding projections, recesses, and combinations thereof of the other component or the acetabulum. In some instances the engagement features are similar to the engagement features of one or more of the prosthetic devices described in U.S. patent application Ser. No. 10/289,126 titled “ONE PIECE SNAP FIT ACETABULAR CUP,” U.S. patent application Ser. No. 10/497,897 titled “CUSHION BEARING IMPLANTS FOR LOAD BEARING APPLICATIONS,” U.S. patent application Ser. No. 10/515,486 titled “IMPLANTS,” U.S. patent application Ser. No. 11/688,153 titled “CERAMIC-ON-CERAMIC PROSTHETIC DEVICE COUPLED TO A FLEXIBLE BONE INTERFACE,” or PCT Application No. PCT/IL2006/000343 titled “IMPLANT DEVICES” (published as WO 2006/097932), each incorporated by reference in its entirety. It is recognized that the various combinations of projections and recesses described as being formed in the acetabulum by these references can instead be formed in the shell component 102 and/or articulating component 104 in accordance with the present disclosure.

In some embodiments, the liner component 104 is formed of a resiliently deformable polymer. In some instances, the liner component 104 is formed of polyurethane. In some instances, the liner component 104 is formed of polycarbonate polyurethane. In some instances, the liner component 104 is formed or polycarbonate polyurethane having a Shore hardness between about 60 Shore A and 100 Shore A and, in one particular embodiment, is about 80 Shore A. In some instances, the liner component 104 is fiber reinforced, includes one or more deformation control elements, and/or comprises a material or combination of materials particularly suited for positioning within an articulating joint. In some embodiments, the liner component 104 is formed of materials or combinations of materials as described in U.S. patent application Ser. No. 10/497,897 titled “CUSHION BEARING IMPLANTS FOR LOAD BEARING APPLICATIONS” and U.S. patent application Ser. No. 12/100,090 titled “MANUFACTURING AND MATERIAL PROCESSING FOR PROSTHETIC DEVICES”, each hereby incorporated by reference in its entirety.

Generally, the shell component 102 is formed of a material that is more rigid than the material of the liner component 104. For example, in some embodiments the shell is formed of a medical grade metal suitable for implantation, including but not limited to stainless steel alloys, cobalt-chrome alloys, titanium alloys, nickel-titanium alloys, and other suitable metals. In other embodiments, the shell is formed of a composite material, including but not limited to polyetheretherketone (PEEK), carbon-reinforced PEEK, Dyneema, and other suitable composites. In some instances, the shell component 102 is formed of a more rigid material than the liner component 104, but the thickness of the shell component is thin enough such that the shell component conforms to the shape of the liner component once the shell component and the liner component are engaged with one another.

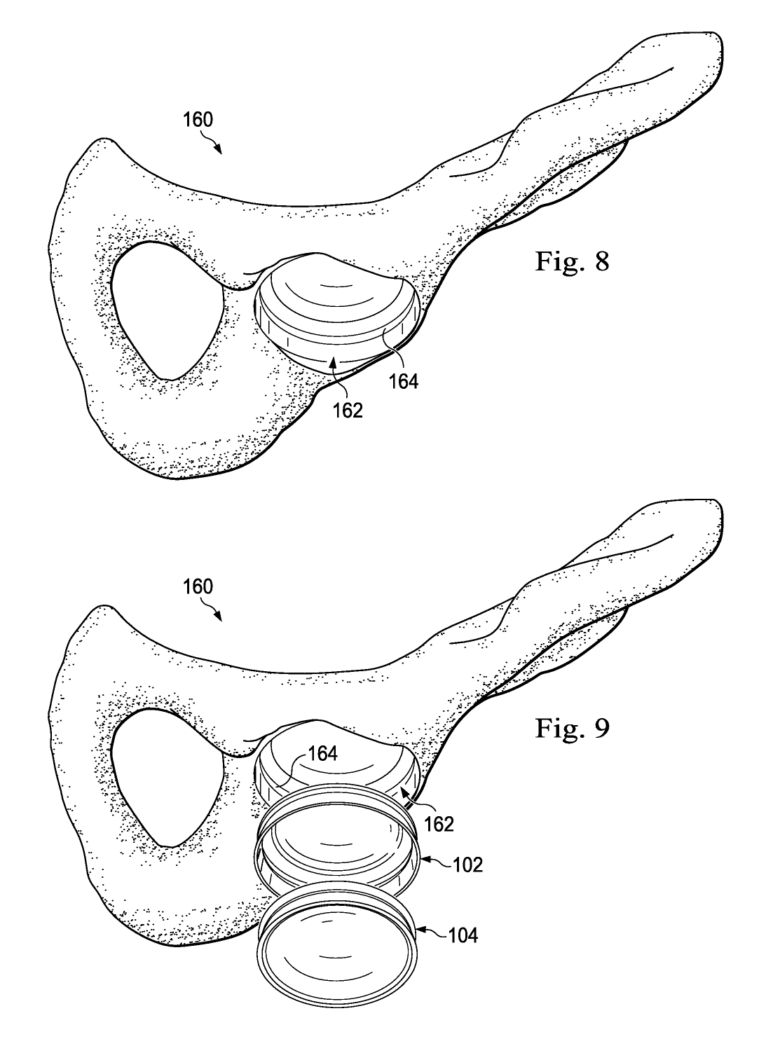

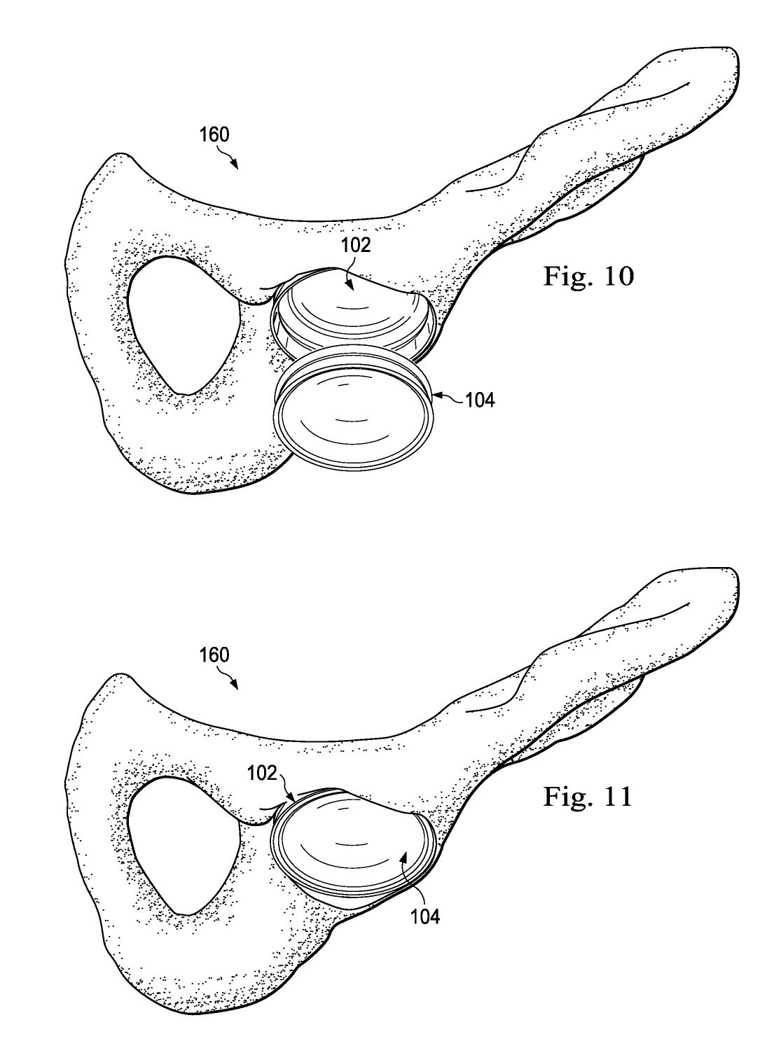

Referring now to FIGS. 8, 9, 10, and 11 shown therein are various stages of the prosthetic device 100 described above being implanted into a patient's acetabulum 160. Specifically, FIG. 8 is a perspective view of a patient's prepared acetabulum 160 according to one aspect of the present disclosure; FIG. 9 is a perspective view of the prosthetic device 100 and the patient's prepared acetabulum 160 illustrating a first stage of implantation; FIG. 10 is a perspective view of the prosthetic device 100 and the patient's prepared acetabulum 106 showing a second stage of implantation; and FIG. 11 is a perspective view of the prosthetic device 100 and the patient's prepared acetabulum 160 showing the prosthetic device fully implanted into the patient's prepared acetabulum.

Referring more specifically to FIG. 8, shown therein is a patient's acetabulum 160. In that regard, a prepared portion 162 of the patient's acetabulum is shown having an annular groove or recess 164. In some instances, preparation of the acetabulum 160 includes reaming a portion of the acetabulum to define the prepared portion 162. In some instances, the prepared portion 162 is partially spherical. In some embodiments, the prepared portion 162 defines a partially spherical surface with a radius of curvature sized for receiving the shell component 102. Generally, the radius of curvature of the prepared portion 162 is sized to substantially match the radius of curvature 122 of the outer surface 106 of the shell component 102. In some particular embodiments, the prepared portion 162 is substantially semi-spherical. In some instances, a bony portion of the acetabulum is reamed or cut to create the prepared portion 162. In that regard, removing at least a portion of the bone can help stimulate bone ingrowth between the prepared portion 162 and the outer surface 106 of the shell component 102 after implantation of the shell component. As shown, the prepared portion 162 also includes the annular groove or recess 164 that is sized and shaped to mate with the anchoring section 114 of the shell component 102 when the anchoring section 114 is in the anchoring configuration. In that regard, the profile of the recess 164 substantially matches that of the anchoring section in some embodiments.

Referring more specifically to FIGS. 9 and 10, after the acetabulum has been prepared the prosthetic device 100, including the shell component 102 and the liner component 104, is implanted into the prepared portion 162 of the acetabulum. As generally illustrated in FIG. 9, the shell component 102 and the liner component 104 are implanted separately in some instances. In that regard, the shell component 102 is inserted into the prepared portion of the acetabulum 162 with the anchoring section 114 in the insertion configuration. After insertion of the shell component 102 into the prepare portion of the acetabulum 162, the anchoring section 114 is transitioned to the anchoring configuration such that the anchoring section engages the recess 164 in the prepared portion of the acetabulum. As shown in FIG. 10, the shell component 102 is fixedly secured within the prepared portion 162 of the acetabulum 160 through the engagement of the anchoring section 114 with the recess 164.

Referring to FIG. 11, after the shell component 102 has been inserted into the prepared portion 162 of the acetabulum 160, the liner component 104 is inserted into the shell component 102. As mentioned above, in some instances insertion of the liner component 104 into the shell component 102 is utilized to transition the anchoring section 114 of the shell component from the insertion configuration to the anchoring configuration. In other instances, the liner component 104 is inserted into the shell component 102 after the anchoring section 114 has been transitioned to the anchoring configuration to fixedly secure the shell component 102 to the prepared portion 160 of the acetabulum 160 via engagement with the recess 164. FIG. 11 illustrates the prosthetic device 100 fully implanted, with the shell component 102 fixedly engaged with the prepared portion 162 of the acetabulum and the liner component 104 fixedly engaged with the shell component 102.

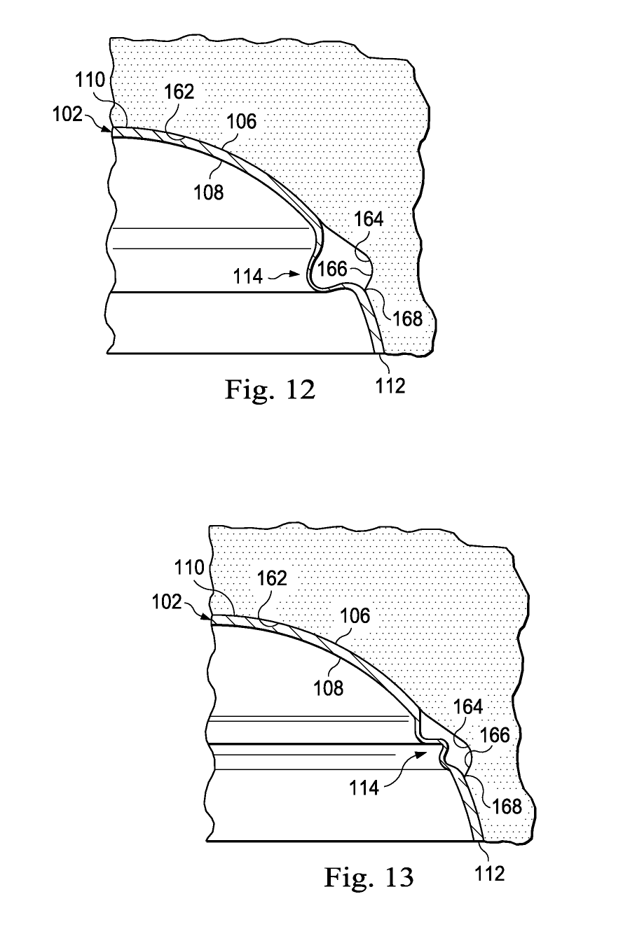

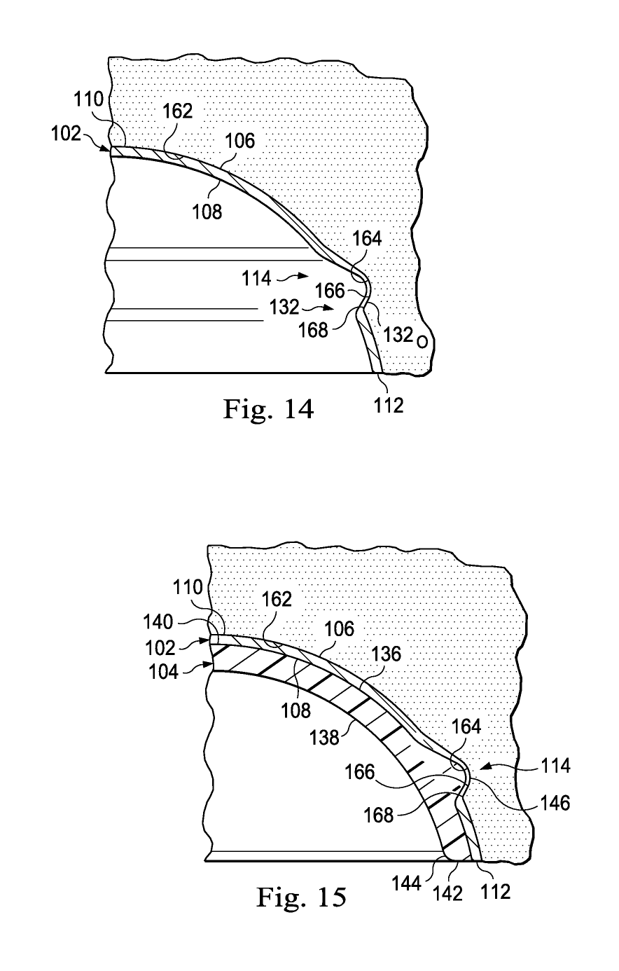

Referring now to FIGS. 12, 13, and 14, illustrated therein is an example of the transition of the anchoring section 114 of the shell component 102 from the insertion configuration to the anchoring configuration to secure the shell component within the prepared portion 162 of the acetabulum 160. Specifically, FIG. 12 is a cross-sectional side view of a portion of the shell component 102 and a section of the prepared portion 162 of the patient's acetabulum 160 with the shell component in the insertion configuration; FIG. 13 is a similar cross-sectional side view but with the shell component in the transition configuration; and FIG. 14 is a similar cross-sectional side view but with the shell component in the anchoring configuration.

Referring more specifically to FIG. 12, when in the insertion configuration the anchoring section 114 projects inward relative to the outer surface 106 such that anchoring section 114 does not engage the recess 164 of the prepared portion 162 of the acetabulum 160 during insertion. In that regard, the recess 164 includes anchoring bone 166. In general, engagement of the anchoring section 114 with the anchoring bone 166 prevents removal of the shell component 102 from the acetabulum. In that regard, the anchoring bone 166 is at least partially defined by a corner 168. The corner 168 is rounded in the illustrated embodiment and generally defines the boundary between the recess 164 and the surrounding area of the prepared portion 162 of the acetabulum. In that regard, in some instances the anchoring portion 166 is a shoulder defined by the corner 168.

Having the anchoring section 114 retracted radially relative to the outer surface also prevents the anchoring section 114 from damaging the area of the prepared portion 162 of the acetabulum 160 adjacent to the recess 164 and, in particular, the corner 168 that could result from inserting the shell component 102 with the anchoring section extending outward from the outer surface 106. In this manner, the shell component 102 is considered corner-preserving. By preserving the structural integrity and geometry of the corner 168, the shell component 102 enhances engagement with the anchoring bone 166 of the recess 164. As shown, the shell component 102 is fully inserted into the prepared portion 162 of the patient's acetabulum 160 such that the apex 140 of the shell component engages an apex of the prepared portion of the acetabulum and the anchoring section 114 is positioned adjacent to the recess 164.

From the insertion configuration of FIG. 12, the anchoring section 114 is moved to the anchoring configuration of FIG. 14. Between the insertion configuration and the anchoring configuration, the anchoring section 114 passes through the transition configuration shown in FIG. 13. As discussed above, the transition of the anchoring section 114 between the insertion configuration and the anchoring configuration is achieved through mechanical force, shape-memory material state change, or other force that results in the profile change. Accordingly, the transition configuration shown in FIG. 13 is only one example of a transition configuration and illustrates only a single point in the transition between the insertion configuration and the anchoring configuration. In that regard, it is understood that the actual shape profile of the anchoring section 114 at any particular point during the transition between the insertion configuration and the anchoring configuration is dependent upon several factors, including but not limited to the structural and/or material properties of the anchoring section 114, the structural and/or material properties of the surrounding portions of the shell component 102, the profile of the anchoring section 114 when in the insertion configuration, the profile of the anchoring section 114 when in the anchoring configuration, and/or the manner in which the anchoring section 114 is being moved between the insertion and anchoring configurations.

Referring now to FIG. 14, the anchoring section 114 has been fully transitioned to the anchoring configuration such that the anchoring section 114 projects outward relative to the outer surface 106. In that regard, in the anchoring configuration the anchoring section 114 defines a projection or protrusion extending from the outer surface 106 that is sized and shaped for secured engagement with the recess 164 in the prepared portion 162 of the acetabulum 160. The engagement between the protrusion defined by the anchoring section 114 and the recess 146 in the acetabulum 160 fixedly secures the shell component 102 within the acetabulum and prevents unwanted loosening and/or removal of the shell component from the acetabulum. In that regard, in addition to the portion 116 of the outer surface 106 defining the anchoring section 114 engaging the recess 164 and the anchoring bone 166, the outer portion 132_{O} of the transition 132 wraps around the corner 168 to help secure the shell component 102 to the acetabulum 160. In addition to defining the projection for engaging the recess 164, the anchoring section 114 also defines a corresponding recess or depression in the inner surface 108 that is sized and shaped for receiving the anchoring projection 146 of the liner component 104.

Referring now to FIG. 15, the liner component 104 has been inserted into the shell component 102. In that regard, the outer surface 136 of the liner component 104 is mated with the inner surface 108 of the shell component 102 such that the anchoring protrusion 146 engages the recess defined by the anchoring section 114 of the shell component 102. In that regard, the anchoring protrusion 146 has a generally rounded profile that is substantially matches the profile of the recess defined by the anchoring section 114 when the anchoring section 114 is in the anchoring configuration. The engagement between outer surface 136 of the liner component 104 and the inner surface 108 of the shell component and, in particular, the engagement of the anchoring protrusion 146 with the recess defined by the anchoring section 114 secures the liner component within the shell component 102 and constrains movement of the liner component relative to the shell component. In some instances, the anchoring protrusion 146 is snap-fit into the recess defined by the anchoring section 114. In that regard, the anchoring protrusion 146 as well as the surrounding portions of the liner component 104 (including the outer and inner surfaces 136, 138) may deform inwardly to facilitate insertion of the liner component into the shell component 102. Once the anchoring protrusion 146 reaches the recess defined by the protrusion, however, the liner component snaps or springs outward so that the anchoring protrusion engages the recess and the liner component 104 is secured to the shell component 102. Though not illustrated in the present embodiment, after the prosthetic device 100 has been fixedly engaged with the prepared portion 162 of the acetabulum 160, a femoral head is mated with the inner surface 138 of the liner component 104 to restore function to the hip joint.

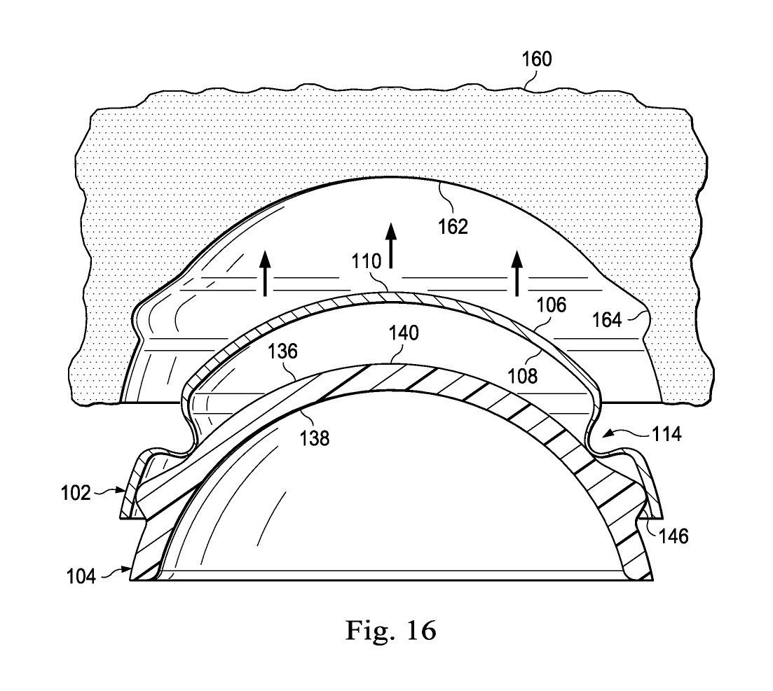

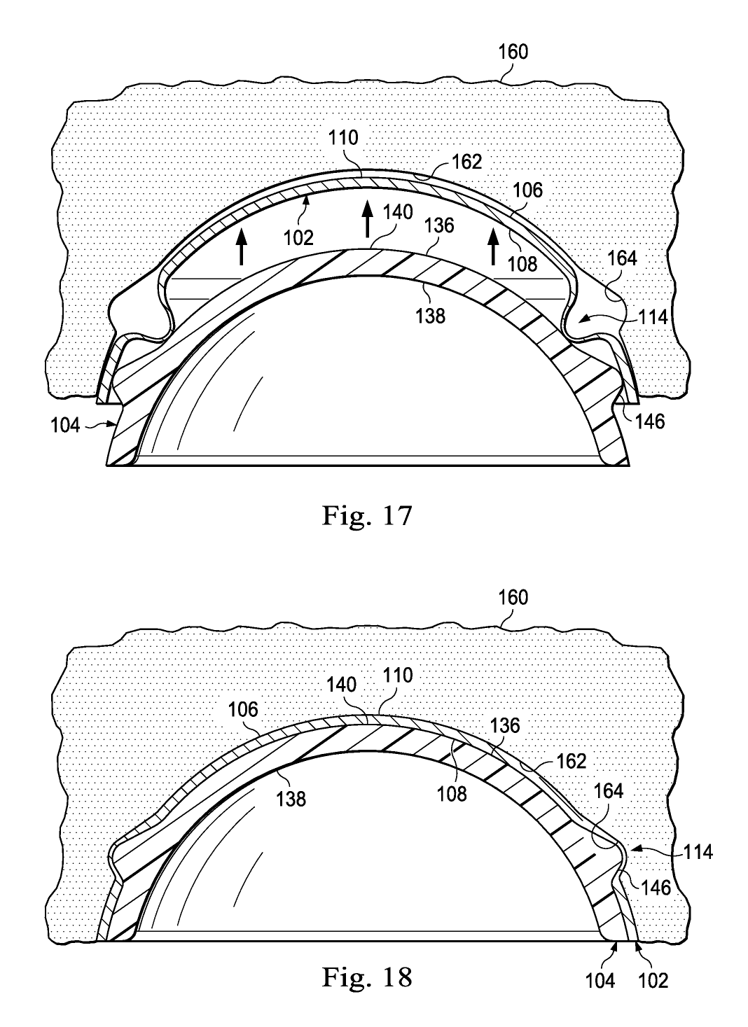

Referring now to FIGS. 16-18, shown therein is a series of drawings illustrating an alternative method of implanting the prosthetic device 100 according to another aspect of the present disclosure. In particular, FIG. 16 is a cross-sectional side view of the prosthetic device 100 and the patient's acetabulum 160 illustrating an initial phase of insertion according to one aspect of the present disclosure; FIG. 17 is a cross-sectional side view of the prosthetic device 100 and the patient's acetabulum 160 illustrating another phase of insertion; and FIG. 18 is a cross-sectional side view of the prosthetic device 100 and the patient's acetabulum 160 illustrating the prosthetic device fully implanted within the patient's acetabulum

The method described above with respect to FIGS. 12-15 illustrated the shell component 102 and the liner component 104 being implanted in two separate steps. In contrast, FIGS. 16-18 illustrate a method of implanting the shell component 102 and the liner component 104 together. In that regard, the liner component 104 is partially inserted into the shell component 102 with the anchoring section 114 in the insertion configuration as shown in FIG. 16. As shown, in this configuration the liner component 104 is inserted into the shell component 102 such that the outer surface 136 of the liner component engages the anchoring section 114 of the shell component and the anchoring protrusion 146 of the liner component engages the lower portion of the inner surface 108 of the shell component. In that regard, the liner component 104 engages the shell component 102 such that the anchoring section 114 is maintained in the insertion configuration. That is, the liner component 104 is not fully inserted into the shell component 102 so that the anchoring section 114 does not protrude beyond the outer surface 106 of the shell component. The shell component 102 and the liner component 104 are maintained in this orientation during initial insertion of the prosthetic device into the acetabulum 160 in some instances. In some embodiments, an insertion tool is utilized to maintain the orientation of the liner component 104 relative to the shell component 102 and/or prevent unwanted advancement of the liner component 104 relative to the shell component 102. In that regard, in some instances the insertion tool engages a portion of each of the shell component 102 and the liner component 104 and maintains them in a spaced relation until the shell component 102 is seated within the prepared acetabulum.

As shown in FIG. 17, the shell component 102 and the liner component 104 are inserted into the prepared portion 162 of the patient's acetabulum such that the apex 140 of the shell component engages an apex of the prepared portion of the acetabulum and the anchoring section 114 is positioned adjacent to the recess 164. In the illustrated embodiment, the shell component 102 and the liner component 104 remain in substantially the same insertion orientation as that shown in FIG. 16. However, in other embodiments, the anchoring section 114 and/or the anchoring protrusion 146 is at least partially deformed relative to the insertion orientation shown in FIG. 16. For example, in some instances, the anchoring section 114 has begun to transition towards the anchoring configuration. In some instances, a portion of the anchoring protrusion 146 is deformed inwardly by the engagement with the anchoring section 114. In that regard, in some embodiments an upper portion of the anchoring protrusion 146 may be deformed inward as the anchoring section 114 initially engages the anchoring protrusion until anchoring section 114 is forced outward by the engagement with the anchoring protrusion.

Referring now to FIG. 18, once the apex 110 of the shell component 102 is engaged with the prepared portion 162 of the acetabulum, the liner component 104 is urged into full engagement with the shell component. In that regard, the liner component 104 is advanced into the shell component 102 until the apex 140 of the liner component engages the inner surface 108 of the shell component. The advancement of the liner component 104 into the shell component causes the anchoring protrusion 146 of the liner component to engage the anchoring section 114 of the shell component 102 and causes the anchoring section to transition from the insertion configuration to the anchoring configuration. In that regard, as the liner component 104 is inserted into the shell component 102 the anchoring section 114 will slide along the outer surface 136 of the liner component until it engages the anchoring protrusion 146, at which point the physical characteristics of the anchoring protrusion will begin exerting a force radially outward on the anchoring section. As the liner component 104 is advanced further into the shell component the radial force imparted on the anchoring section 114 through engagement with the anchoring protrusion causes the anchoring section to deform outward to the anchoring configuration. As shown, the transition of the anchoring section 114 to the anchoring configuration results in secure engagement of the anchoring section with the recess 164 in the acetabulum 160, which thereby secures the shell component 102 to the acetabulum. The transition of the anchoring section 114 to the anchoring configuration also results in the anchoring protrusion 146 of the liner component 104 being fixedly engaged with the resulting recess defined by the anchoring section, which thereby secures the liner component 104 to the shell component 102. Accordingly, by advancing the liner component 104 into the shell component 102, the shell component 102 is secured to the prepared portion 162 of the acetabulum 160 and the liner component 104 is secured to the shell component, as shown in FIG. 18.

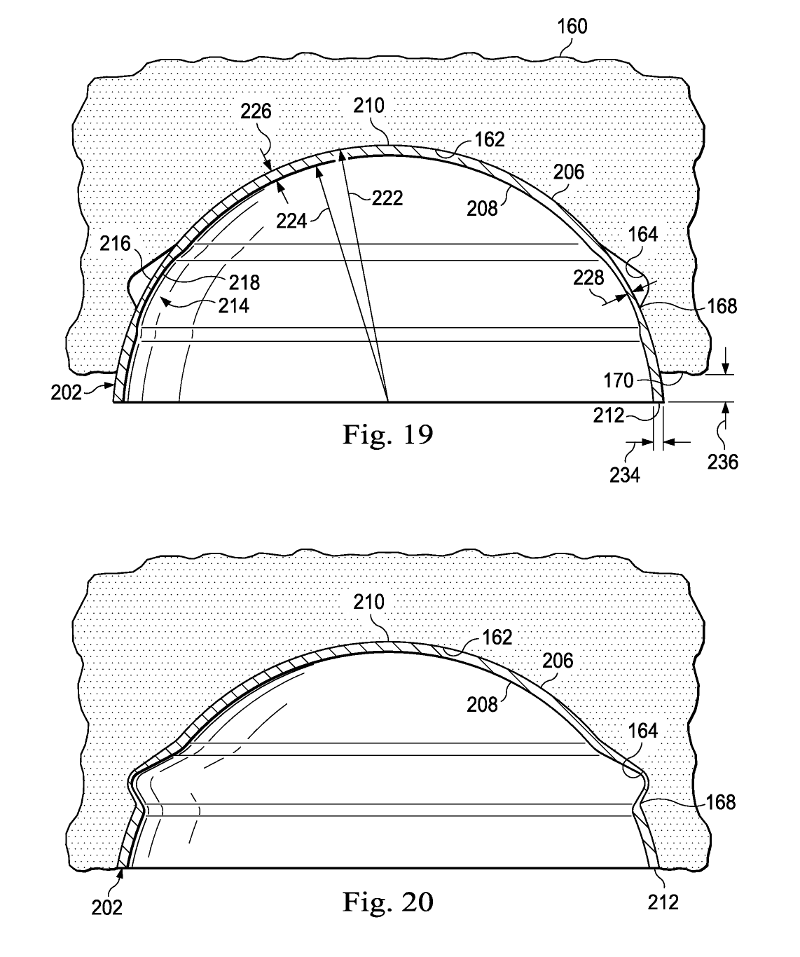

Referring now to FIGS. 19 and 20, shown therein is a FIG. 19 is a cross-sectional side view of a shell component 202 within a patient's prepared acetabulum 160 according to another aspect of the present disclosure. In particular, FIG. 19 illustrates the shell component 202 in an insertion configuration, while FIG. 20 illustrates the shell component 202 in an anchored configuration. As shown, the shell component 202 includes an outer surface 206 for engaging a prepared portion 162 of the patient's acetabulum 160. In that regard, in some instances, the outer surface 206 of the shell component 202 is treated to enhance engagement between the outer surface and the patient's acetabulum 160. Further, in some instances the outer surface 206 includes structural features for encouraging engagement between the shell component 202 and the acetabulum 160. Opposite the outer surface 206 the shell component 202 includes an inner surface 208. The shell component 202 has an apex 210 and a rim 212.

In the illustrated embodiment, the shell component 202 includes an anchoring section 214. Referring more specifically to FIG. 19, the shell component 202 is shown in an insertion configuration. In the insertion configuration, a majority of the outer surface 206 has a generally semi-spherical profile and a majority of the inner surface 208 has a generally semi-spherical profile that is concentric with the semi-spherical profile of the outer surface 206. From a center point 220, the majority of the outer surface 206 is defined by a radius of curvature 222, while the majority of the inner surface 208 is defined by a radius of curvature 224. As illustrated, the majority of the outer surface 206 and the majority of the inner surface 208 are separated by a thickness 226. Generally, the thickness 226 is between about 0.1 mm and about 1.5 mm and, in some embodiments, is between about 0.25 mm and about 1.25 mm. In one particular embodiment, the thickness is approximately 0.75 mm. The radius of curvature 222 is generally between about 20 mm and about 34 mm, but in some instances may be larger or smaller. Accordingly, the radius of curvature 224 is generally between about 19.0 mm and about 33.9 mm, but also may be larger or smaller in some instances.

In some embodiments, the outer surface 106 and/or the inner surface 108 has a profile that extends less than or more than a semi-spherical profile. For example, in some instances the profile extends between about 160 degrees and 179.9 degrees. In some instances, where the profile extends beyond a semi-spherical profile (i.e., 180 degrees), the portion of the profile extending beyond the semi-spherical profile is generally planar. That is, the extended portion extends substantially parallel to a tangent of the outer surface at the boundary of the semi-spherical profile. In that regard, in some instances the extended portion is retracted during transition of the shell component 202 between an insertion configuration and anchoring configuration such that, in the anchoring configuration, the extended portion is retracted such that the shell component has a generally semi-spherical profile between the apex 210 and the rim 212.

The shell component 202 includes an anchoring section 214. As shown, the anchoring section 214 is defined by a portion 216 of the outer surface 206 and a portion 218 of the inner surface 208. Generally, the anchoring section is deformable or movable between the insertion configuration (illustrated in FIG. 19) and an anchoring configuration (illustrated in FIG. 20). In the illustrated embodiment, the anchoring section 214 has a thickness 228 between the portions 216, 218 of the outer and inner surfaces 206, 208 defining the anchoring section. The thickness 228 of the anchoring section 214 is less than or equal to the thickness 226. The thickness 228 is typically between about 0.1 mm and about 1.5 mm and, in some embodiments, is between about 0.1 mm and about 1.0 mm. In one particular embodiment, the thickness is approximately 0.3 mm. In that regard, in some instances the shell component 202 includes a transition between the anchoring section 214 and the upper portion of the shell component positioned between the anchoring section and the apex 210. In that regard, the transition tapers the thickness of the shell 202 from thickness 226 to thickness 228. In the illustrated embodiment, the thickness 228 is illustrated as being approximately half of the thickness 226. The transition is illustrated as being a gradual taper, but in other embodiments the taper is steeper and, in some instances, is abrupt such that an edge or step is defined by the change in thickness. In some instances the transition to the anchoring section 214 is defined by a change in the material properties of the shell component 202. In that regard, in some embodiments, the thickness 228 of the anchoring section 214 is substantially equal to the thickness 226, but the material properties of the anchoring section 214 are different from those of at least the upper portion of the shell component 202 between the anchoring section and the apex 110. In some embodiments, the transition includes both a change in thickness as well as a change in material properties.

Similarly, the shell component 202 includes a transition between the anchoring section 214 and the lower portion of the shell component positioned between the anchoring section and the rim 212. In that regard, the transition tapers the thickness of the shell 202 from thickness 228 to a thickness 234 between the outer surface 206 and the inner surface 208 adjacent the rim 212. The thickness 234 is generally equal to or greater than the thickness 226 and, therefore, is also generally equal to or greater than the thickness 228. In that regard, the thickness 234 is typically between about 0.1 mm and about 1.5 mm and, in some embodiments, is between about 0.25 mm and about 1.25 mm. In one particular embodiment, the thickness is approximately 0.75 mm.

In some instances, the thickness 234 of the lower portion of the shell component 202 is increased relative to the thickness 228 of the anchoring section 214 to prevent unwanted deformation of the shell component, or at least the rim 212, during transition or deformation of the anchoring section from the insertion configuration of FIG. 19 to the anchoring configuration of FIG. 20. In other instances, the shell component 202 includes a reinforcing ring or band around, within, and/or inside the lower portion of the shell component to limit deformation of the shell component between the anchoring section 214 and the rim 212. In some instances, the material properties of the lower portion of the shell component 202 are different from those of at least the anchoring section 214. Thus, in some embodiments the transition between the anchoring section 214 and the lower portion of the shell component 202 is defined by a change in the material properties of the shell component 202. In that regard, in some embodiments, the thickness 234 is substantially equal to the thickness 228 of the anchoring section 214, but the material properties of the anchoring section 214 are different from those of at least the lower portion of the shell component 102 between the anchoring section and the rim 212. In some embodiments, the transition includes both a change in thickness as well as a change in material properties. In the illustrated embodiment, the thickness 234 is illustrated as being approximately twice the thickness 228. Accordingly, the transition is illustrated as a taper that facilitates the change in thickness. The transition is illustrated as being a gradual taper, but in other embodiments the taper is steeper and, in some instances, is abrupt such that an edge or step is defined by the change in thickness.

As noted above, the anchoring section 214 is movable between an insertion configuration where the portion 216 of the outer surface 206 defining the anchoring section generally conforms to the semi-spherical profile of the majority of the outer surface (as shown in FIG. 19) and an anchoring configuration where the portion 216 of the surface 206 defining the anchoring section outward relative to the majority of the outer surface of the shell component (as shown in FIG. 20). Referring more specifically to FIG. 19, the insertion configuration of the anchoring section 214 facilitates safe insertion of the shell component 202 into a prepared acetabulum. In that regard, if the shell component 202 was inserted with the anchoring section 214 protruding radially outward from the outer surface, the anchoring section could cause damage to the acetabulum as the shell component is urged into the acetabulum. By having the anchoring section 214 generally aligned with the profile of the majority of the outer surface in the insertion configuration, the potential for damaging the prepared acetabulum during insertion of the shell component 202 is greatly reduced or eliminated completely. As shown in FIG. 19, when the shell component 202 is inserted into the acetabulum 160 in the insertion configuration, with the apex 210 positioned against the prepared portion 162 of the acetabulum the rim 212 of the shell component extends beyond an outer surface 170 of the acetabulum by a distance 236 as shown. In some instances, the distance 236 is between about 0.1 mm and about 5.0 mm, but may be greater in some instances. In other instances, the rim 212 of the acetabulum is positioned within the boundary defined by the outer surface 170.

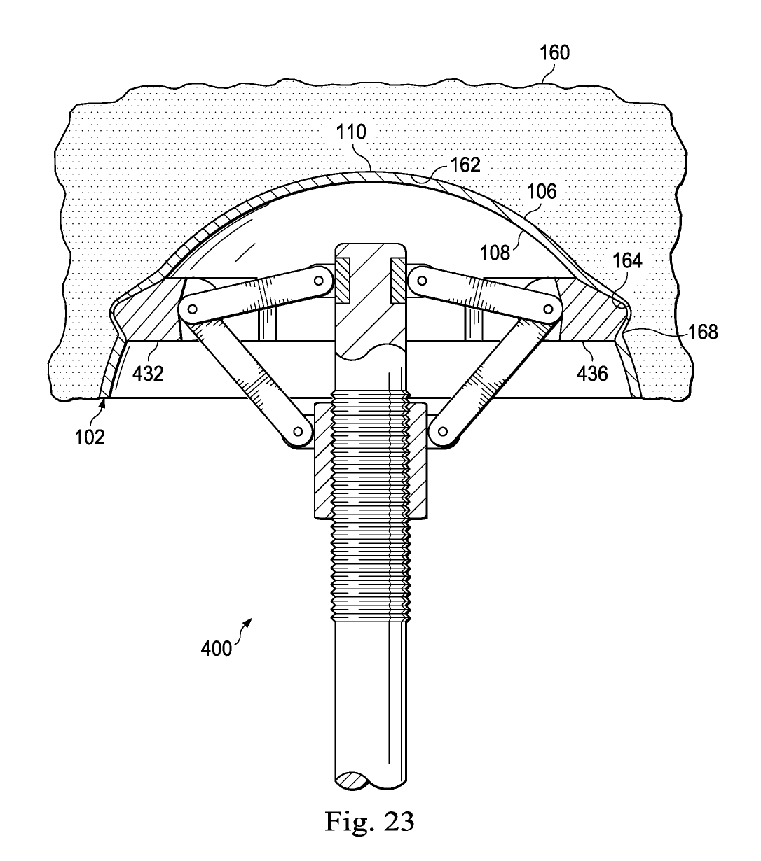

From the insertion configuration, the anchoring section 214 is moved to the anchoring configuration. The transition between the insertion configuration and the anchoring configuration is achieved through mechanical force in some instances. For example, a mechanical tool is utilized to urge the anchoring section 214 from the insertion configuration to the anchoring configuration. Generally, the tool will exert a radially outward force on the anchoring section 214, which causes the anchoring section to deform from the insertion configuration to the anchoring configuration. In that regard, the tools discussed below with respect to FIGS. 23-27 are examples of tools suitable for transitioning the anchoring section 214 between the insertion and anchored configurations. However, any tool capable of transitioning the anchoring section 214 from the insertion configuration to the anchored configuration may be utilized.

In other instances, the transition between the insertion configuration and the anchoring configuration is achieved by transitioning a shape-memory material between a first material state and a second material state. In that regard, the anchoring section 214 comprises a shape-memory material, such as Nitinol, in some embodiments. The shape-memory material is configured such that in the first state the shape-memory material is biased to the insertion configuration and in the second state the shape-memory material is biased to the anchoring configuration. In some instances, the transition of the shape-memory material between the first and second states and, thereby, the insertion and anchoring configurations is achieved by changing the temperature of the shape-memory material. In some embodiments, the shape-memory material is biased towards the second state (i.e., the anchoring configuration) when the shape-memory material is between about 35° C. and about 40° C.

Referring more specifically to FIG. 20, once the anchoring section 214 has been fully transitioned to the anchoring configuration the anchoring section 214 projects outward relative to the outer surface 206 such that it defines projection or protrusion extending from the outer surface 206 and defines a corresponding recess or depression in the inner surface 208. In that regard, in some instances the anchoring section 214 is particularly suited to fixedly engage a recess 164 prepared in the acetabulum 160. The engagement between the protrusion defined by the anchoring section 214 and the recess 164 in the acetabulum 160 fixedly secures the shell component 202 within the acetabulum and prevents unwanted loosening and/or removal of the shell component from the acetabulum.

Transitioning the anchoring section 214 from the insertion configuration to the anchored configuration causes a retraction of the rim 212 relative to the outer surface 170 of the acetabulum 160. Specifically, the deformation of the anchoring section 214 outward pulls the rim 212 towards the apex 210 of the shell component 202. The retraction of the rim 212 results in the rim extending beyond the outer surface 170 a distance less than the distance 236. In some instances, the retraction of the rim 212 results in the rim being substantially aligned with the outer surface 170 of the acetabulum 160. In other instances, the retraction of the rim 212 results in the rim being positioned inside the boundary defined by the outer surface 170 of the acetabulum 160. Though not illustrated in the present embodiment, a liner component, such as liner component 104 described above, is utilized in conjunction with shell component 202 in some instances.

Referring now to FIG. 21, shown therein is a cross-sectional side view of an insertion tool 300 being utilized to transition the shell component 102 positioned within the patient's prepared acetabulum 160 from an insertion configuration to a transition configuration according to another aspect of the present disclosure. In that regard, the tool 300 includes a working distal portion 302 and an elongated main shaft 304 extending therefrom. The tool includes a proximal portion (not shown) opposite the distal portion 302. In some instances the proximal portion includes a handle graspable by a user. As shown, the elongated main shaft 304 includes a threaded portion 306 and a distal end 308. Threadingly mated with the threaded portion 306 of the main shaft 302 is a threaded sleeve 310. As discussed below, the threaded sleeve 310 is movable along a longitudinal axis 312 with respect to the main shaft 304. Pivotally attached to the sleeve 310 are arms 314 and 316. In that regard, arm 314 is attached at a pivot point 318, while arm 316 is attached at a pivot point 320. Further, an arm 322 is pivotally attached to the main shaft 302 at pivot point 324, while an arm 326 is pivotally attached to the main shaft 302 at pivot point 328. In that regard, the pivot points 324 and 328 are in a fixed relationship relative to the distal end 308 of the shaft 302, whereas the pivot points 318 and 320 that are attached to the sleeve 310 are movable relative to the distal end 308 of the shaft 302. As shown, arms 314 and 322 are pivotally connected to one another at a pivot point 330. Further, a bumper 332 is movably joined to the arms 314, 322 at the pivot point 330. As shown, the bumper 332 includes a surface that is sized and shaped to mate with the anchoring section 114 of the shell 102 when the shell is in the insertion configuration. In particular, the bumper 332 includes a pair of arcuate protrusions that are spaced by a concave recess. In some instances, the concave recess has a radius of curvature that substantially matches that of the anchoring section 114 such that the anchoring section mates with the concave recess of the bumper 332 (as shown in FIG. 21). In some instances, the arcuate protrusions surrounding the concave recess help to maintain the anchoring section 114 engaged with the bumper 332 and, in particular, the recess. Further, in some instances at least one of the arcuate protrusions is shaped to urge the anchoring section 114 into the anchored configuration.

Similarly, arms 316 and 326 are pivotally connected to one another at a pivot point 334. A bumper 336 is movably joined to the arms 316, 326 at the pivot point 334. As shown, the bumper 336 is similar to bumper 332 and includes a surface that is sized and shaped to mate with the anchoring section 114 of the shell 102 when the shell is in the insertion configuration. In particular, the bumper 336 includes a pair of arcuate protrusions that are spaced by a concave recess. In some instances, the concave recess has a radius of curvature that substantially matches that of the anchoring section 114 such that the anchoring section mates with the concave recess of the bumper 336 (as shown in FIG. 21). In some instances, the arcuate protrusions surrounding the concave recess help to maintain the anchoring section 114 engaged with the bumper 336 and, in particular, the recess.

While the tool 300 is illustrated in FIG. 19 as having two sets of arms 314,322 and 316, 326, it is understood that the tool 300 actually has a total of four pair of arms equally spaced about the circumference of the shaft 302. In that regard, each pair of arms is associated with a corresponding bumper, such that the tool 300 has four bumpers that will engage the anchoring section 114 of the shell component 102. However, it is understood that any number of sets of arms may be utilized in a similar manner. For example, in some embodiments 2, 3, 5, 6, 7, 8, 9, and 10 sets of arms are utilized. Similarly, any number of bumpers may be utilized. In that regard, in some instances, more than one set of arms are connected to a single bumper. Accordingly, the function of the tool 300 described below with respect to arms 314, 316, 322, 326 and bumpers 332, 336 will be understood to apply similarly to other combinations of sets of arms and bumpers.

In use, the tool 300 is inserted into the shell 102 such that the bumpers 332, 336 engage the anchoring section 114 of the shell. In that regard, the tool 300 is inserted into the shell 102 and the shaft 302 is rotated to cause movement of the sleeve 310 along the longitudinal axis 312 of the shaft to facilitate engagement of the bumpers 332, 336 with the anchoring section 114. In that regard, rotation of the shaft 302 clockwise as viewed from the proximal end of the shaft, as indicated by arrow 338, results in the sleeve 310 moving proximally along the longitudinal axis 312, as indicated by arrow 340, which in turn causes the bumpers 332, 336 to be retracted radially inward, as indicated by arrow 342. On the other hand, rotation of the shaft 302 counter-clockwise as viewed from the proximal end of the shaft, as indicated by arrow 344, results in the sleeve 310 moving distally along the longitudinal axis 312, as indicated by arrow 346, which in turn causes the bumpers 332, 336 to be extended radially outward, as indicated by arrow 348. It is understood that if the threads were reversed, the relative movements would similarly be reversed.

In some instances the tool 300 is sized to match a particular size shell 102. For example, for a particular sized shell 102, the dimensions of the anchoring section 114 in the insertion configuration are known. Accordingly, the bumpers of the tool 300 can be arranged to match the orientation of the anchoring section 114. In one such embodiment, the bumpers of the tool match the orientation of the anchoring section 114 when the bumpers are in a fully retracted position. Further, in some instances the tool 300 includes an index or markings on the shaft 302 to facilitate engagement of the bumpers with the anchoring structure of a particular size of shell. For example, in some instances alignment of a portion of the sleeve 310 with a marking will be indicative of the size of shell 102 that the bumpers are currently arranged to interface with based on the position of the sleeve. In some instances, the markings are color-coded to the shell sizes.

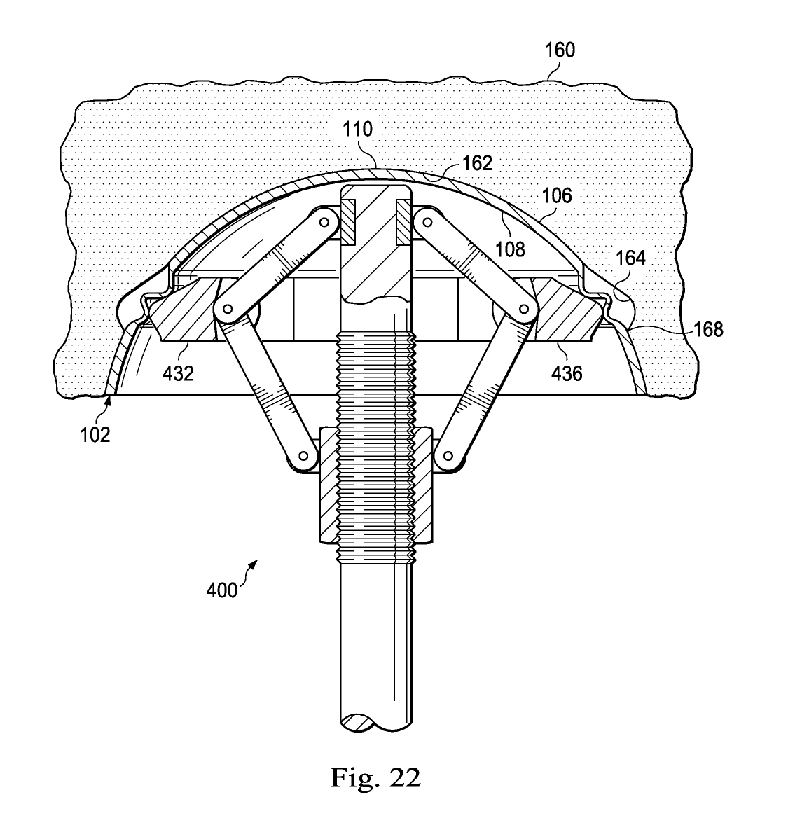

Once the bumpers 332, 336 are engaged with the anchoring section 114 of the shell, the shaft 302 is rotated to cause the bumpers to be expanded radially outward. As the bumpers 332, 336 are expanded radially outward the anchoring section 114 is similarly transitioned outward towards the anchoring configuration. In some instances, the bumpers 332, 336 are initially used to transition the anchoring section 114 to an intermediate configuration, as indicated by phantom profile 350. In some instances, a second tool is utilized to transition the anchoring section 114 from the intermediate configuration to the anchoring configuration (e.g., see FIGS. 22 and 23 and corresponding description below). In other instances, the bumpers 332, 336 themselves are utilized to transition the anchoring section 114 to the anchoring configuration. In one such embodiment, after reaching the intermediate configuration the bumpers are slightly retracted by rotation of the shaft 302 such that one of the pair of protrusions of the bumpers engage the anchoring section. In that regard, the convex profile of the arcuate protrusions helps to facilitate the full transition to the anchoring configuration in some instances.

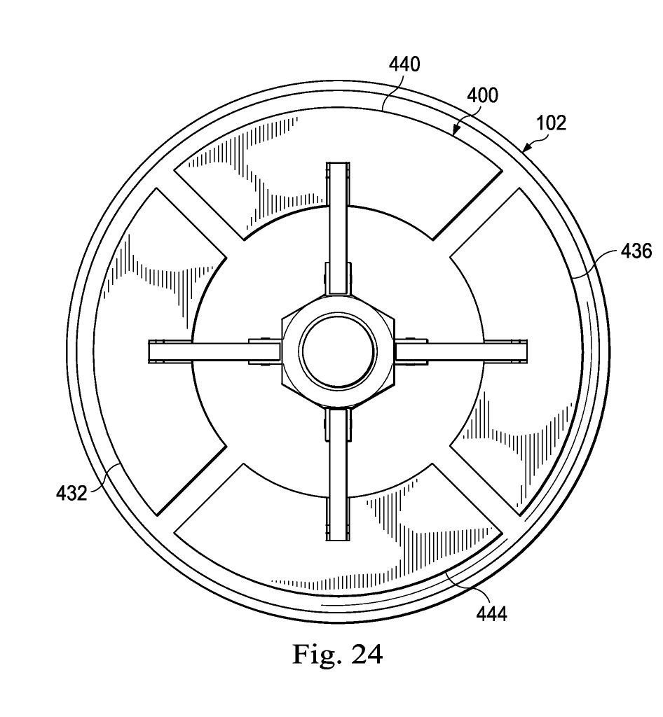

Referring now to FIGS. 22-24, shown therein is another insertion tool 400 being utilized to transition the shell component 102 positioned within a patient's prepared acetabulum from the transition configuration to an anchored configuration according to another aspect of the present disclosure. In that regard, the tool 400 is substantially similar to tool 300 described above except that tool 400 includes bumpers 432, 436, 440, and 444 (see FIG. 24) particularly sized and shaped to facilitate transition of the anchoring section 114 to the anchored configuration. In that regard, as shown in FIGS. 22 and 23, the bumpers 432, 436 include convex outer surfaces that generally match the shape of the inner surface of the anchoring section 114 when the anchoring section is in the anchored configuration. Accordingly, the bumpers 432, 436 are particularly suited to drive the anchoring section 114 from the transition configuration to the desired anchored configuration. In that regard, in some instances the tool 400 is utilized in combination with the tool 300 to move the anchoring section 114 from the insertion configuration to the anchored configuration, with the tool 300 moving the anchoring section from the insertion configuration to an intermediate configuration and the tool 400 moving the anchoring section from the intermediate configuration to the anchored configuration. In some instances, the tool 400 is utilized to transition the anchoring section 114 from the insertion configuration to the anchored configuration. As a general matter, while the tools 300 and 400 have been described with respect to shell 102 and anchoring section 114, the tools 300 and 400 are similarly suitable for use with the shell 202 and its anchoring section 214. In particular, the tools 300 and 400 are suitable to transition the anchoring section 214 from the insertion configuration of FIG. 19 to the anchored configuration of FIG. 20.

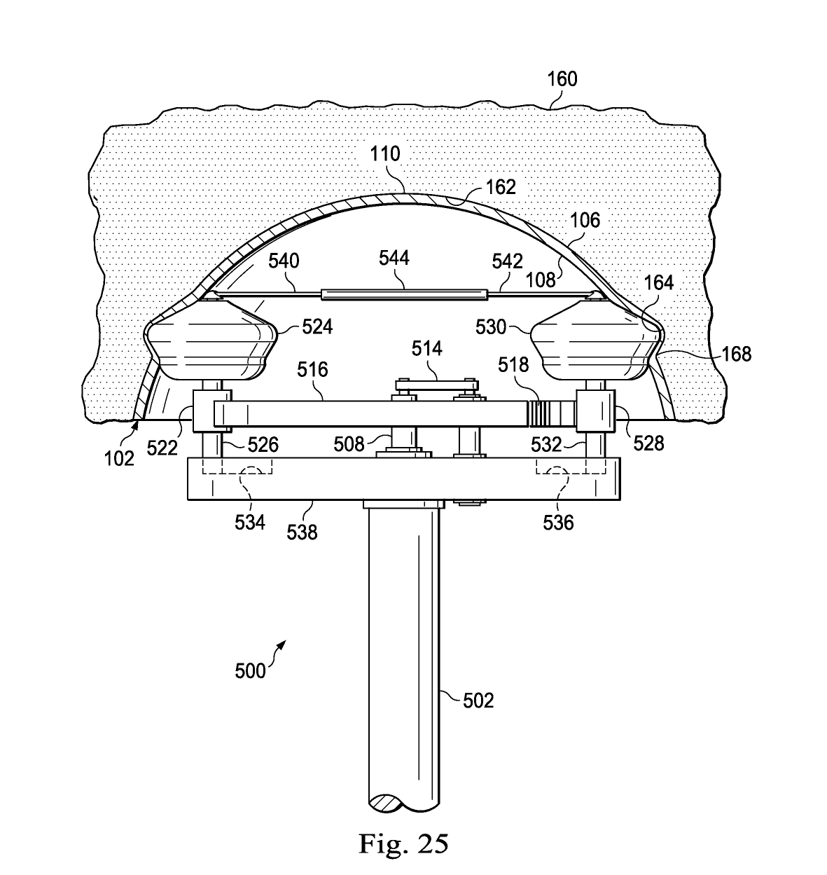

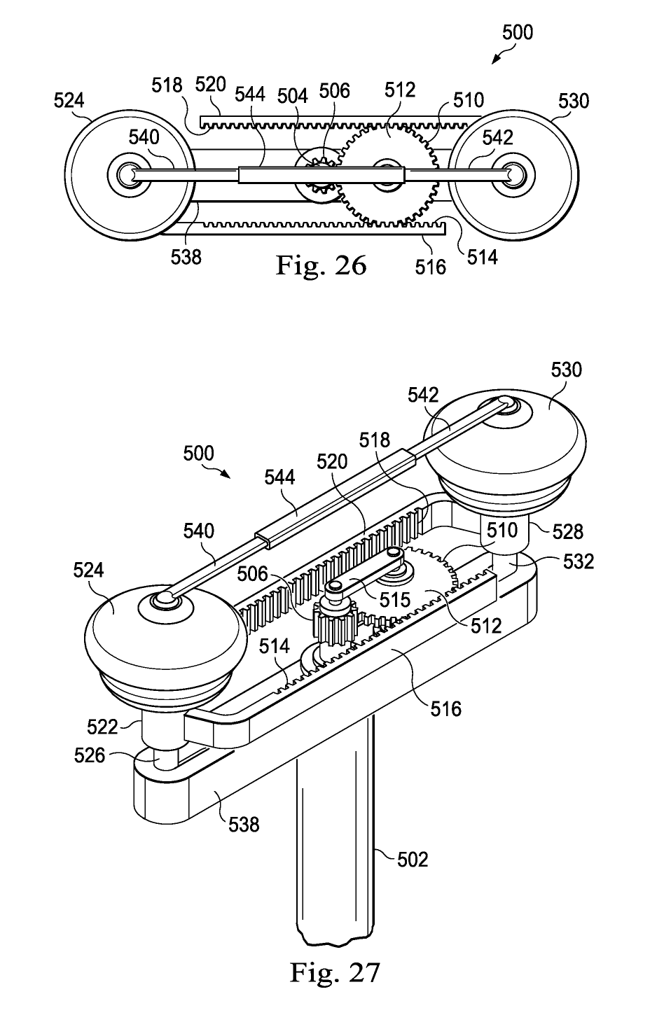

Referring now to FIGS. 25-27, shown therein is an insertion tool 500 that is utilized to transition a shell component 102 positioned within a patient's prepared acetabulum from an insertion configuration to an anchored configuration according to another aspect of the present disclosure. In that regard, FIG. 25 is a cross-section side view of the tool 500 engaged with the shell 102 positioned within the prepared acetabulum 160, FIG. 26 is a bottom view of the tool 500, and FIG. 27 is a perspective view of the tool. As a general matter, the tool 500 is utilized to exert an outward radial force on the anchoring section of the shell component to transition the anchoring section into an anchored configuration. In that regard, the tool 500 is suitable for use with anchoring sections such as anchoring sections 114 and 214 described above with respect to shells 102 and 202. For simplicity, the tool 500 will be discussed in the context of shell 102 and anchoring section 114.

As shown, the tool 500 includes an elongated shaft 502 has a proximal portion and distal portion. In some instances, the proximal portion includes a handle for grasping by a user. Associated with the distal portion of the shaft 502 is a wheel 504 having gears 506. In the illustrated embodiment, the wheel 504 is connected to an inner shaft 508 extending along the length of the shaft 502. In that regard, in some embodiments the inner shaft 508 includes a handle adjacent the proximal portion of the shaft 502 that is rotatable to cause a corresponding rotation of the wheel. In some instances, the handle of the inner shaft 508 is configured for engagement with another tool, including powered (electrical and pneumatic) and simple mechanical tools, that assists in rotating the shaft and/or provides a desired amount of force to rotation of the wheel 504. In some instances, the wheel 504 is fixedly attached to the shaft 502 such that rotation of the shaft 502 itself causes a corresponding rotation of the wheel 504.

The gears 506 of the wheel 504 are engaged with gears 510 of a wheel 512. The pivot points of the wheels 504 and 512 are maintained in fixed relationship at least in part by bar 514 extending therebetween. In addition to engaging with gears 506 of the wheel 504, the gears 510 of wheel 512 also engage gears 514 of elongated bar 516 and gears 518 of elongated bar 520. In that regard, the engagement of the wheel 512 with the elongated bars 516, 520 generally acts as a rack and pinion system, where rotation of the wheel 512 causes corresponding linear movement of the bars 516, 520. Bar 516 in turn is connected to a support 522 that is associated with a roller 524 that revolves around a post 526 extending through the roller. Similarly, bar 520 is connected to a support 528 that is associated with a roller 530 that revolves around a post 532 extending through the roller. Bottom portions of the posts 526 and 532 are positioned within recesses 534, 536 of a guide member 538, respectively. In that regard, the recesses 534, 536 assure that the posts 526, 532 travel along a generally linear path when the wheel 504 is rotated. In doing so, the engagement between the posts 526, 532 and the recesses 534, 536 also prevents unwanted rotational movement of the bars 516 and 520 associated with the posts. Further, upper portions of the posts 526, 532 are connected by guide bars 540, 542 and sleeve 544. In that regard, the sleeve 544 is an integral part of one of the guide bars 540, 542 in some instances. In other instances, the sleeve 544 is a separate component.

In use, the tool 500 is inserted into the shell 102 such that the rollers 524, 530 engage the anchoring section 114 of the shell. In that regard, the tool 500 is inserted into the shell 102 and the inner shaft 508 is rotated to cause rotation of the wheel 504, which in turn causes linear movement of bars 516, 520 and, thereby, rollers 524, 530 to facilitate engagement of the rollers with the anchoring section 114. In that regard, rotation of the shaft 508 clockwise as viewed from the proximal end of the shaft results in the bars 516, 520 being displaced outward relative to the shaft 502, which in turn causes the rollers 524, 530 to be similarly extended outward. On the other hand, rotation of the inner shaft 508 counter-clockwise as viewed from the proximal end of the shaft results in the bars 516, 520 retracted inward toward the shaft 502, which in turn causes the rollers 524, 530 to be similarly retracted inward. It is understood that if the gearing was reversed, the relative movements would similarly be reversed.

Once the rollers 524, 530 are engaged with the anchoring section 114 of the shell, the shaft 502 is rotated relative to the shell 102 so that the rollers 524, 530 rotate around the inside of the shell contacting the different circumferential portions of the anchoring section. In some instances, the inner shaft 508 is simultaneously rotated with the shaft 502 such that the rollers are expanded outward as they rotate around the anchoring section 114 of the shell 102. In other instances, the rollers 524, 530 are positioned at a first expansion distance and the shaft 502 is rotated, then the rollers 524, 530 are expanded outward to a second expansion distance and the shaft 502 is rotated again. In some instances, the shaft 502 is rotated approximately 180 degrees between expansions of the rollers 524, 530. As the rollers 524, 530 are expanded outward and rotated about the shell 102 (either simultaneously or step-wise), the anchoring section 114 is urged outward towards the anchoring configuration. Generally, the rollers 524, 530 are expanded outward and rotated about the shell 102 until the anchoring section is fully expanded to the anchoring configuration and secured to the acetabulum (as shown in FIG. 25).

In some instances the tool 500 is sized to match a particular size shell 102. For example, for a particular sized shell 102, the dimensions of the anchoring section 114 in the insertion configuration are known. Accordingly, the rollers of the tool 500 can be arranged to match the orientation of the anchoring section 114. In one such embodiment, the rollers of the tool match the orientation of the anchoring section 114 when the bumpers are in a fully retracted position. Further, in some instances the tool 500 includes an index or markings on the guide member 538 to facilitate engagement of the rollers with the anchoring structure of a particular size of shell. For example, in some instances alignment of a portion of bar 516 or bar 520 with a marking will be indicative of the size of shell 102 that the rollers are currently arranged to interface with based on the position of the bar. In some instances, the markings are color-coded to the shell sizes. In some instances, the tool 500 includes markings for both an initial engagement with the anchoring section and an anchored engagement with the anchoring section. For example, the tool 500 includes a first marker associated with the orientation necessary for the initial engagement with the anchoring section and a second marker that indicates the maximum expanded for the rollers for a particular sized shell. In other instances, the tool 500 includes a fully retracted position that corresponds to the initial engagement position and a fully extended position that corresponds to the anchored engagement position.

The foregoing outlines features of several embodiments so that those skilled in the art may better understand the aspects of the present disclosure. Those skilled in the art should appreciate that they may readily use the present disclosure as a basis for designing or modifying other processes and structures for carrying out the same purposes and/or achieving the same advantages of the embodiments introduced herein. Those skilled in the art should also realize that such equivalent constructions do not depart from the spirit and scope of the present disclosure, and that they may make various changes, substitutions, and alterations to the embodiments disclosed herein without departing from the spirit and scope of the present disclosure. Also, it will be fully appreciated that variations of the above-disclosed and other features and functions, or alternatives thereof, may be combined into other methods, systems, apparatus, or applications. Similarly, various presently unforeseen or unanticipated alternatives, modifications, and/or variations of the present disclosure subsequently made by those skilled in the art are also encompassed by the present disclosure and the following claims.

1. A prosthetic device for implantation into a hip joint comprising:
a metal shell component comprising a convex outer surface for securely engaging a prepared portion of an acetabulum and an opposing concave inner surface for receiving a polymer liner component, a majority of the outer surface having a generally semi-spherical profile, a majority of the inner surface having a generally semi-spherical profile concentric with the outer surface, at least a portion of the inner and outer surfaces defining an anchoring section extending circumferentially about the metal shell component between an apex of the metal shell component and a rim of the metal shell component, the anchoring section deformable between an insertion configuration and an anchoring configuration, wherein in the insertion configuration the anchoring section projects inwardly from the inner surface and wherein in the anchoring configuration the anchoring section projects outwardly from the outer surface to define an anchoring protrusion for engaging the prepared portion of the acetabulum, wherein a majority of the metal shell component has a substantially uniform thickness between the outer surface and the inner surface and wherein the anchoring section has a thickness between the outer surface and the inner surface that is less than the substantially uniform thickness of the majority of the metal shell component; and
the polymer liner component comprising an outer surface having a generally semi-spherical profile for engagement with the inner surface of the metal shell component, the outer surface comprising an annular protrusion extending circumferentially about the outer surface, the annular protrusion sized and shaped to engage a recess defined by the inner surface of the anchoring section of the metal shell component when the anchoring section is in the anchoring configuration, the polymer liner component further comprising an inner surface for articulatingly mating with a femoral head.

2. The prosthetic device of claim 1, wherein the substantially uniform thickness of the metal shell component is between about 0.1 mm and about 3.0 mm.

3. The prosthetic device of claim 2, wherein the polymer liner component has a thickness greater than the substantially uniform thickness of the metal shell component.

4. The prosthetic device of claim 3, wherein the thickness of the polymer liner component is at least twice the thickness of the metal shell component.

5. The prosthetic device of claim 1, wherein in the insertion configuration the anchoring section does not extend radially outward beyond the generally semi-spherical profile of the majority of the outer surface.

6. The prosthetic device of claim 1, wherein in the insertion configuration the anchoring section defines a convex annular protrusion extending inwardly from the generally semi-spherical profile of the majority of the inner surface.

7. The prosthetic device of claim 1, wherein in the anchoring configuration the anchoring section does not extend radially inward beyond the generally semi-spherical profile of the majority of the inner surface.

8. The prosthetic device of claim 1, wherein at least the anchoring section of the metal shell component comprises a shape-memory alloy.

9. The prosthetic device of claim 8, wherein the anchoring section is deformable between the insertion configuration and the anchoring configuration by a transition of the shape-memory alloy.

10. The prosthetic device of claim 9, further comprising a prosthetic femoral head for mating with the inner surface of the liner component.

11. A method comprising:
preparing a portion of an acetabulum to receive a hip prosthesis;
obtaining a hip prosthesis comprising:
a metal shell component comprising a convex outer surface for securely engaging the prepared portion of the acetabulum and an opposing concave inner surface for receiving a polymer liner component, a majority of the outer surface having a generally semi-spherical profile, a majority of the inner surface having a generally semi-spherical profile concentric with the outer surface, at least a portion of the inner and outer surfaces defining an anchoring section extending circumferentially about the metal shell component between an apex of the metal shell component and a rim of the metal shell component, the anchoring section deformable between an insertion configuration and an anchoring configuration, wherein in the insertion configuration the anchoring section projects inwardly from the inner surface and wherein in the anchoring configuration the anchoring section projects outwardly from the outer surface; and
the polymer liner component comprising an outer surface having a generally semi-spherical profile for engagement with the inner surface of the metal shell component, the outer surface comprising an annular protrusion extending circumferentially about the outer surface, the polymer liner component also having an inner surface for articulatingly mating with a femoral head;
inserting the metal shell component of the hip prosthesis into the prepared portion of the acetabulum with the anchoring section in the insertion configuration;
deforming the anchoring section of the metal shell component from the insertion configuration to the anchoring configuration to securely fix the metal shell component to the prepared portion of the acetabulum by engagement of the anchoring section with the prepared portion of the acetabulum; and
inserting the polymer liner component such that the outer surface of the polymer liner component engages the inner surface of the metal shell component.

12. The method of claim 11, wherein inserting the polymer liner component such that the outer surface of the polymer liner component engages the inner surface of the metal shell component the anchoring section of the metal shell component to deform from the insertion configuration to the anchoring configuration.

13. The method of claim 11, wherein deforming the anchoring section of the metal shell component from the insertion configuration to the anchoring configuration includes engaging a tool with the metal shell component and rotating at least a portion of the tool relative to the metal shell component.

14. The method of claim 11, wherein preparing the portion of the acetabulum includes creating a recess and wherein deforming the anchoring section of the metal shell component from the insertion configuration to the anchoring configuration to securely fix the metal shell component to the prepared portion of the acetabulum includes engaging the anchoring section with the recess.

15. The method of claim 11, wherein inserting the polymer liner component includes snap-fitting the annular protrusion of the polymer liner with a recess defined by the anchoring section of the metal shell component when the anchoring section is in the anchoring configuration.

16. The method of claim 11, wherein deforming the anchoring section of the metal shell component from the insertion configuration to the anchoring configuration includes transitioning a shape-memory alloy from a first state to a second state thereby causing the anchoring section to move from the insertion configuration to the anchoring configuration.

17. A method of implanting an artificial acetabular component comprising:
inserting a metal shell into a prepared acetabulum, wherein the metal shell includes a convex outer surface and an opposing concave inner surface, wherein a majority of the outer surface has a partially spherical profile and a majority of the inner surface has a partially spherical profile substantially concentric with the majority of the outer surface, wherein at least a portion of the inner and outer surfaces define an anchoring section, the anchoring section deformable between an insertion configuration and an anchoring configuration, wherein in the insertion configuration the anchoring section projects inwardly relative to the partially spherical profile of the outer surface and wherein in the anchoring configuration the anchoring section projects outwardly relative partially spherical profile of the outer surface, wherein the metal shell is inserted into the prepared acetabulum with the anchoring section in the insertion configuration; and
deforming the anchoring section of the metal shell component from the insertion configuration to the anchoring configuration to secure the metal shell to the prepared acetabulum by engagement of the anchoring section with the prepared acetabulum.

18. The method of claim 17, wherein deforming the anchoring section of the metal shell from the insertion configuration to the anchoring configuration includes engaging a tool with the metal shell and rotating at least a portion of the tool relative to the metal shell.

19. The method of claim 18, wherein deforming the anchoring section of the metal shell from the insertion configuration to the anchoring configuration includes transitioning a shape-memory alloy from a first state to a second state thereby causing the anchoring section to move from the insertion configuration to the anchoring configuration.

20. The method of claim 19, further comprising creating a recess in the acetabulum, and wherein deforming the anchoring section of the metal shell from the insertion configuration to the anchoring configuration to secure the metal shell component to the prepared acetabulum includes engaging the anchoring section with the recess.
